(19) United States
(12) Patent Application Publication
(10) Pub. No.: US 2026/0224483 A1
(43) Pub. Date: Aug. 6, 2026

(54) STABLE ORAL LIQUID PHARMACEUTICAL COMPOSITIONS OF LURASIDONE

(71) Applicant: AZURITY PHARMACEUTICALS, INC., Woburn, MA (US)

(72) Inventors: Paras Jain, Amaravati District (IN); Krishna Mohan Lakshmipathula, Hyderabad (IN); Dadapeer Shaik, Jammalamadugu (IN); Sandeep Jain, Makronia (IN); Satheesh Balasubramanian, Hyderabad (IN); Sumitra Ashokkumar Pillai, Hyderabad (IN)

(73) Assignee: AZURITY PHARMACEUTICALS, INC., Woburn, MA (US)

(21) Appl. No.: 19/460,201

(22) Filed: Jan. 26, 2026

(30) Foreign Application Priority Data
Jan. 27, 2025 (IN) 202541006643

(51) Int. Cl. A61K 9/10; A61K 9/00; A61K 31/496; A61K 47/02; A61K 47/10; A61K 47/12; A61K 47/26; A61K 47/34; A61K 47/38; A61K 47/46

(52) U.S. Cl. CPC A61K 9/10; A61K 9/0053; A61K 31/496; A61K 47/02; A61K 47/10; A61K 47/12; A61K 47/26; A61K 47/34; A61K 47/38; A61K 47/46

(57) ABSTRACT

Stable oral liquid pharmaceutical compositions of lurasidone or pharmaceutically acceptable salts thereof are disclosed, along with methods of administration, processes for their production, and use of these compositions for treatment of CNS disorders in a human, including schizophrenia and depressive episodes associated with bipolar disorder.

### CROSS-REFERENCE TO RELATED APPLICATIONS

This application claims priority to Indian Application No. IN 202541006643, filed on Jan. 27, 2025, which is incorporated herein by reference in its entirety.

### FIELD OF THE INVENTION

The present disclosure relates to stable oral liquid pharmaceutical compositions of lurasidone or pharmaceutically acceptable salts thereof, methods for their administration, processes for their manufacture/production, and use of these compositions for treatment of CNS disorders in a human, including schizophrenia and depressive episodes associated with bipolar disorder.

### BACKGROUND OF THE INVENTION

Lurasidone is an antagonist with high affinity binding at the dopamine D2 receptors, serotonin 5-HT2A and 5-HT7 receptors. Lurasidone has the chemical name N-4-4-(1,2-benzisothiazol-3-yl)-1-piperazinyl-(2R,3R)-2,3-tetramethylene-butyl-(1R,2S,3R,4'S)-2,3-bicyclo[2.2.1]heptane-dicarboxyimide, and has the structural formula (I), shown below:

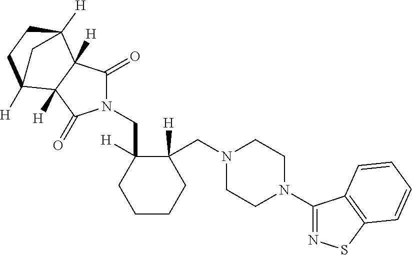

Lurasidone is currently marketed in the United States under the brand name LATUDA®, and is provided as 20 mg, 40 mg, 60 mg, 80 mg and 120 mg tablets for oral administration. Lurasidone hydrochloride is a white to off-white powder. It is very slightly soluble in water, practically insoluble or insoluble in 0.1 N HCl, slightly soluble in ethanol, sparingly soluble in methanol, practically insoluble or insoluble in toluene and very slightly soluble in acetone.

As lurasidone is very slightly soluble in water, attaining sufficient bioavailability of this drug is problematic. It is estimated that about 9-19% of an administered dose is absorbed in the fed state. LATUDA® must be administered with food (at least 350 calories), which substantially increases the absorption. For example, LATUDA® mean Cmax and AUC values are increased by about 3-times and 2-times, respectively, when administered with food compared to the levels observed under fasting conditions.

Although the commercially available solid dosage form of LATUDA® is meant for treating adult as well as for pediatric population, they lack ease-of-use for pediatric population or persons with dysphagia due to several factors, such as the ability to swallow or palatability issues. Thus, there is a need in the art to provide an altermate dosage form of LATUDA® which is advantageous in comparison to the commercially available solid oral dosage form of LATUDA®.

Because of the problems associated with commercially available solid oral dosage forms of LATUDA®, it is desirable to develop stable liquid compositions of lurasidone suitable for oral administration to human subjects, which are ready-to-use and safe-to-administer, allow flexibility in administration of doses, are therapeutically effective, and exhibit prolonged room temperature stability.

### SUMMARY OF THE INVENTION

The present invention fulfils such a need by developing stable oral liquid compositions of lurasidone to achieve an improved standard of patient care.

In some aspects, the present invention relates to stable oral liquid pharmaceutical compositions of lurasidone or its pharmaceutically acceptable salts thereof, for treatment of CNS disorders in humans, including schizophrenia and depressive episodes associated with bipolar disorder. The inventive lurasidone oral compositions are advantageously ready-to-use (RTU) and/or safe-to-administer (STA).

In some aspects, the present invention provides oral liquid pharmaceutical compositions of lurasidone having extended stability, while maintaining a potency appropriate for a pharmaceutical dosage form.

In some aspects, the present invention relates to stable oral liquid pharmaceutical compositions comprising lurasidone or its pharmaceutically acceptable salt thereof and one or more pharmaceutically acceptable excipients, wherein the concentration of lurasidone ranges from about 5 mg/mL to about 50 mg/mL.

Some aspects relate to a stable oral liquid pharmaceutical composition comprising:
(a) lurasidone or a pharmaceutically acceptable salt thereof;
(b) at least one pharmaceutically acceptable liquid vehicle; and
(c) optionally, one or more excipients selected from group consisting of suspending agents, preservatives, buffering agents, wetting agents, anti-foaming agents, anti-caking agents, bitter blockers, thickening agents, pH modifiers, anti-oxidants, complexing agents, chelating agents, flavoring agents, sweetening agents, coloring agents or combinations thereof;
wherein the pH of the pharmaceutical composition is in a range from about 1.5 to about 5; and
wherein the pharmaceutical composition is stable for at least 1 month when stored at about 40° C. and about 75% relative humidity (RH).

Some aspects relate to the stable liquid pharmaceutical composition, wherein the composition is a suspension or a solution. In some aspects, the composition is an aqueous suspension.

In some aspects, the pH of the composition ranges from about 2 to about 3.

In some aspects, the buffering agent is present at a concentration that ranges from about 0.1% w/v to about 5% w/v.

In some aspects, the buffering agent is present, and the buffering agent comprises citric acid monohydrate, anhydrous citric acid, disodium hydrogen phosphate sodium citrate, sodium acetate, sodium phosphate, potassium phosphate, tris, sodium succinate, sodium tartrate, sodium benzoate, gluconic acid, ascorbic acid or a combination thereof.

In some aspects, the suspending agent is present, and wherein a concentration of the suspending agent is in a range of about 0.01% to about 10% w/v.

In some aspects, the suspending agent is present, and wherein the suspending agent comprises xanthan gum, guar gum, acacia gum, gellan gum, carboxymethylcellulose sodium and hydroxy ethylcellulose, microcrystalline cellulose, hydroxypropyl methylcellulose, sodium alginate, methylcellulose, colloidal silica or a combination thereof.

In some aspects, the suspending agent is present, and the ratio of lurasidone or salt thereof to the suspending agent is in a range of about 1:0.5 to about 1:10.

In some aspects, the preservative is present, and the concentration of the preservative is in a range of 0.1% to about 5% w/v.

In some aspects, the preservative is present and the preservative comprises methyl paraben, propyl paraben, sodium propionate, benzyl alcohol, potassium sorbate, sodium benzoate, butylated hydroxyanisole, butylated hydroxytoluene, chlorobutanol, phenyl ethyl alcohol or a combination thereof.

In some aspects, the wetting agent is present and the concentration of the wetting agent is in a range of 0.01% to about 5% w/v.

In some aspects, the wetting agent comprises poloxamer (P188), polysorbate 80, polyethylene glycols (PEGs), sorbitan monolaurate, polysorbate 20, sodium lauryl sulfate, sorbitan esters, lecithin, or a combination thereof.

In some aspects, the pharmaceutically acceptable liquid vehicle comprises water, ringer's solution, isotonic sodium chloride solution, glycerin, propylene glycol, ethanol, polyethylene glycol or a combination thereof.

In some aspects, the anti-foaming agent is present and the anti-foaming agent comprises simethicone, alcohols, paraffin oils, stearates and glycols or a combination thereof.

In some aspects, the anti-caking agent is present, and the concentration of the anti-caking agent is in a range of 0.01% to about 10% w/v.

In some aspects, the anti-caking agent is present, and the anti-caking agent comprises colloidal silica, magnesium trisilicate, calcium phosphate tribasic, magnesium oxide, magnesium silicate, calcium silicate, talc or a combination thereof.

In some aspects, the sweetening agent is present, and the concentration of the sweetening agent is in a range of 0.01% to about 3% w/v.

In some aspects, the sweetening agent is present, and the sweetening agent comprises sucralose, trehalose, xylose, dextrose, tagatose, glycerol, lactitol, sodium saccharine, sodium cyclamate, aspartame, acesulfame or a combination thereof.

In some aspects, the bitter blocker is present, and the bitter blocker comprises adenosine 5′-monophosphate, homoeriodictyol sodium, 3β-hydroxydihydrocostunolide, gingerdione, or a combination thereof.

Some aspects relate to an stable oral suspension comprising:
(a) about 0.01% to about 5% w/v of lurasidone or a pharmaceutically acceptable salt thereof,
(b) at least one suspending agent;
(c) at least one buffering agent;
(d) at least one pharmaceutically acceptable vehicle; and
(e) optionally, at least one pharmaceutically acceptable excipient selected from preservatives, wetting agents, anti-foaming agents, anti-caking agents, bitter blockers, thickening agents, pH modifiers, stabilizers, anti-oxidants, chelating agents, flavoring agents, sweetening agents, coloring agents, or combination thereof, and
wherein, as measured using a USP Apparatus II at a paddle rotation speed of 50 RPM in 900 mL of a dissolution medium at 37±0.5° C., at least about 70 wt % of lurasidone dissolves within about 15 minutes in pH 3.8 McIlvaine buffer dissolution medium;
wherein the pH of the suspension ranges from about 2.0 to about 6.0; and
wherein the suspension is stable after being stored at about 45° C. and about 75% RH for at least 1 month.

In some aspects, the lurasidone or the pharmaceutically acceptable salt thereof has a D90 particle size of about 10 μm or less.

In some aspects, the suspension is a ready-to-use composition that does not need further reconstitution prior to administration.

In some aspects, the suspension comprises lurasidone or a pharmaceutical acceptable salt thereof at a concentration in a range of about 0.05 mg/mL to about 20 mg/mL.

In some aspects, the oral suspension comprises lurasidone or a pharmaceutical acceptable salt thereof at a concentration of about 10 mg/mL.

In some aspects, the present invention relates to stable oral liquid pharmaceutical compositions comprising lurasidone or its pharmaceutically acceptable salt thereof and one or more pharmaceutically acceptable excipients selected from solubilizing agents, salt forming agents, suspending agents, wetting agents, anti-foaming agents, thickening agents, pH modifiers, buffering agents, stabilizers, anti-oxidants, chelating agents, preservatives, flavoring agents, sweetening agents, anticaking agents, bitter blockers, coloring agents, or mixtures thereof.

In some aspects, the present invention relates to oral liquid pharmaceutical compositions comprising lurasidone fumarate and one or more pharmaceutically acceptable excipients selected from solubilizing agents, salt forming agents, suspending agents, wetting agents, anti-foaming agents, thickening agents, pH modifiers, buffering agents, stabilizers, anti-oxidants, chelating agents, preservatives, flavoring agents, sweetening agents, coloring agents or mixtures thereof.

In some aspects, the present invention relates to stable oral solution comprising lurasidone fumarate and one or more pharmaceutically acceptable excipients selected from solubilizing agents, salt forming agents, suspending agents, wetting agents, anti-foaming agents, thickening agents, pH modifiers, buffering agents, stabilizers, anti-oxidants, chelating agents, preservatives, flavoring agents, sweetening agents, coloring agents, or mixtures thereof.

In some aspects, the present invention relates to stable oral suspension comprising lurasidone fumarate and one or more pharmaceutically acceptable excipients selected from solubilizing agents, salt forming agents, suspending agents, wetting agents, anti-foaming agents, thickening agents, pH modifiers, buffering agents, stabilizers, anti-oxidants, chelating agents, preservatives, flavoring agents, sweetening agents, coloring agents or mixtures thereof.

In some aspects, the present invention relates to stable oral liquid composition comprising lurasidone fumarate; wherein lurasidone fumarate is formed in situ. In another aspect, the present invention relates to oral liquid composition comprising lurasidone or its pharmaceutically acceptable salt thereof, wherein the lurasidone salt is formed in situ by dissolving lurasidone in a solubilizing agent and then reacting the dissolved lurasidone with a molar excess of a salt forming agent. In some embodiments, lurasidone salt which is formed in situ is selected from lurasidone fumarate, lurasidone tartrate, lurasidone tosylate, lurasidone mesylate, lurasidone succinate, lurasidone salicylate, lurasidone maleate, lurasidone acetate, or mixtures thereof. In some aspects, these salts exhibit enhanced solubility in aqueous medium compared to lurasidone free base or lurasidone hydrochloride.

In some aspects, the present invention relates to stable oral liquid pharmaceutical composition comprising lurasidone and a salt forming agent; wherein the ratio of lurasidone to salt forming agent in the composition is in a range from about 1:1 to about 1:5.

In some aspects, the present invention relates to stable oral liquid pharmaceutical composition comprising lurasidone and optionally a salt forming agent; wherein a ratio of lurasidone to salt forming agent in the composition ranges from about 1:1 to about 1:5.

In some aspects, the present invention relates to stable oral liquid pharmaceutical composition comprising lurasidone or its pharmaceutically acceptable salts thereof, the stable oral liquid composition comprises lurasidone at a concentration ranging from about 1 mg/mL to about 200 mg/mL; and at least one pharmaceutically acceptable vehicle, wherein the composition remains stable when stored at 40° C.±2° C./75%±5% RH or 30° C.±2° C./65%±5% RH or 25° C.±2° C./60% RH or 40° C.±2° C./25%±5% RH for at least 3 months or at 2-8° C. for at least 6 months.

In some aspects, the present invention relates to stable oral liquid pharmaceutical composition comprising lurasidone fumarate and one or more pharmaceutically acceptable excipients; wherein the stable oral liquid pharmaceutical composition is stable for at least 6 months when stored at 25° C.±2° C./60%±5% RH or 40° C.±2° C./75%±5% RH or 30° C.±2° C./65% 5% RH or 40° C. 2° C./25% 5% RH and the level of impurities in the composition is less than 2% w/w as measured by HPLC.

In some aspects, the present invention relates to stable oral liquid pharmaceutical composition comprising lurasidone or its pharmaceutically acceptable salts thereof, wherein the particle size of lurasidone in the composition is in the range of about 0.1 to about 150 microns.

In some aspects, the present invention relates to stable oral liquid pharmaceutical composition comprising lurasidone or pharmaceutically acceptable salts thereof, wherein the composition is a suspension or a solution.

In some aspects, the present invention specifically relates to oral solution of lurasidone, wherein the oral solution is a palatable solution, which remains stable for extended periods of time, when stored at 40° C.±2° C./75%±5% RH or 30° C.±2° C./65%±5% RH or 25° C.±2° C./60% RH or 40° C.±2° C./25%±5% RH.

Some aspects relate to oral suspension of lurasidone, wherein the oral suspension is a palatable formulation, which remains stable for extended periods of time, when stored at 40° C.±2° C./75%±5% RH or 30° C.±2° C./65%±5% RH or 25° C.±2° C./60% RH or 40° C.±2° C./25%±5% RH.

In some aspects, the present invention provides a stable solution for oral administration comprising: (i) lurasidone; (ii) salt forming agent; and (iii) optionally, one or more pharmaceutically acceptable excipients.

In some aspects, the present invention provides a stable solution for oral administration comprising: (i) lurasidone; (ii) salt forming agent; (iii) at least one pharmaceutically acceptable vehicle; and (iv) optionally, one or more pharmaceutically acceptable excipients.

In some aspects, the present invention provides a stable suspension for oral administration comprising: (i) lurasidone; (ii) salt forming agent; and (iii) optionally, one or more pharmaceutically acceptable excipients.

In some aspects, the present invention provides a stable suspension for oral administration comprising: (i) lurasidone; (ii) salt forming agent; (iii) at least one pharmaceutically acceptable vehicle; and (iv) optionally, one or more pharmaceutically acceptable excipients.

By way of non-limiting examples, exemplary combinations applicable to the embodiments described in this application may include any combination with one or more of the elements described above.

### DETAILED DESCRIPTION OF THE INVENTION

Unless defined otherwise, all the technical and scientific terms used herein have the same meanings as commonly known to a person of ordinary skill in the art. In case of conflict, the definitions provided herein will prevail.

Unless specified otherwise, all the percentages, portions and ratios in the present invention are on weight basis.

Unless otherwise indicated, all numbers expressing quantities of ingredients, properties such as molecular weight, reaction conditions, and so forth used in the present specification and associated claims are to be understood as being modified in all instances by the term “about.” The terms “about” and “approximate,” when used along with a numerical variable, generally means the value of the variable and all the values of the variable within a measurement or an experimental error (e.g., 95% confidence interval for the mean) or within a specified value (e.g., ±10% or ±20%) within a broader range.

Accordingly, unless indicated to the contrary, the numerical parameters set forth in the following specification and attached claims are approximations that may vary depending upon the desired properties sought to be obtained by the embodiments of the present invention. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values.

Also, the terms in the claims have their plain, ordinary meaning unless otherwise explicitly and clearly defined by the patentee.

Moreover, the indefinite articles “a” or “an,” as used in the claims, are defined herein to mean one or more than one of the elements that it introduces. As used in the description herein and throughout the claims that follow, the meaning of “a,” “an,” and “the” includes plural reference unless the context clearly dictates otherwise.

While compositions and methods are described herein in terms of “comprising” various components or steps, the compositions and methods can also “consist essentially of” or “consist of” the various components and steps.

As used herein the term “lurasidone” refers to lurasidone free base or its pharmaceutically acceptable salts, solvates or hydrates, or derivatives or isomers thereof. In principle, any crystalline form or amorphous form of lurasidone may be used for manufacturing the inventive pharmaceutical compositions. In embodiments, pharmaceutically acceptable salt of lurasidone is selected from lurasidone hydrochloride, lurasidone fumarate, lurasidone tartrate, lurasidone tosylate, lurasidone mesylate, lurasidone succinate, lurasidone salicylate, lurasidone maleate, lurasidone acetate, or mixtures thereof.

The term “pharmaceutically acceptable” substances mean those, which, according to a common medical judgment, are suitable to be in contact with a tissue of a patient without any inappropriate toxicity, irritation, allergic response, etc., have a reasonable balance between advantages and disadvantages, and can be applied to its target use effectively.

The term “pharmaceutically acceptable salt” refers to salts which are formed with inorganic or organic acids and are pharmaceutically acceptable.

The terms “composition,” “pharmaceutical composition,” and “formulation” are used interchangeably herein, and refer to a combination of two or more ingredients. For example, a composition may comprise an active ingredient (e.g., lurasidone) and a suspending agent. In some embodiments, the composition is in the form of a liquid.

The term “liquid pharmaceutical composition” refers to a pharmaceutical composition orally administered to a patient, including a solution or a suspension.

The term “ready-to-use” as used herein, refers to a formulation that does not require constitution or dilution with a prescribed amount of diluent, e.g., purified water or other suitable diluent, before use by the designated route.

The term “safe-to-administer” as used herein, refers to a formulation which is non-toxic and safe for humans, in the proposed concentrations and total doses used.

The terms “dosage”, “dose unit” or “dose” as used herein means the amount of a pharmaceutical formulation comprising therapeutically active ingredient (s) administered at a time.

By “effective amount” or “therapeutically effective amount” is meant the amount of a drug sufficient to treat, prevent, or ameliorate a condition in a subject or patient. The effective amount of lurasidone or its pharmaceutically acceptable salts thereof, may be determined and adjusted by a person of ordinary skill to provide the appropriate amount and dosage regimen, e.g., depending upon manner of administration, the age, body weight, sex, and/or general health of the patient.

The term “solubility” means solubility of lurasidone or its pharmaceutically acceptable salts in media such as water, aliphatic or aromatic alcohols, oils, surfactants, polyols, buffer, gastrointestinal simulated fluid, gastrointestinal fluid and the like.

The term “subject” refers to an animal, including a human or non-human. The terms patient and subject may be used interchangeably herein.

The terms “stable” and “stability” mean that the evolution of the product with time and/or under specific environmental conditions (i.e., temperature, humidity, etc.) has no significant effects on its quality, safety and/or efficacy for a given time period. It can be measured through the formation of degradation products (impurities), variation of pH, microbial growth, or physical appearance, such as precipitation and/or color.

The term “any person” refers to any human being capable of administering dose of lurasidone composition to a patient, wherein human being includes physicians, healthcare professions, nurse, pharmacist, pharmacy technicians and the patient.

As used herein, “to treat” a condition or “treatment” of the condition is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable.

The term “concentration” refers to the amount of a component in a composition. The term “concentration” is used interchangeably herein with “amount.” The term “concentration” when used herein in reference to lurasidone or a pharmaceutically acceptable salt thereof should be understood to be referring to the concentration of free base, rather than the salt.

As used in, the terms “level” and “amount” when referring to impurities may be used interchangeably and refer to a total concentration of the impurity or impurities in a composition.

The present application relates to stable liquid pharmaceutical compositions of lurasidone or its pharmaceutically acceptable salts thereof, wherein lurasidone is present at a concentration ranging from about 1 mg/mL to about 200 mg/mL.

In some embodiments, the concentration of lurasidone or its pharmaceutically acceptable salt thereof in the inventive liquid pharmaceutical compositions is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, about 9.9 mg/mL, about 10 mg/mL, about 10.1 mg/mL, about 10.2 mg/mL, about 10.3 mg/mL, about 10.4 mg/mL, about 10.5 mg/mL, about 10.6 mg/mL, about 10.7 mg/mL, about 10.8 mg/mL, about 10.9 mg/mL, about 11 mg/mL, about 11.1 mg/mL, about 11.2 mg/mL, about 11.3 mg/mL, about 11.4 mg/mL, about 11.5 mg/mL, about 11.6 mg/mL, about 11.7 mg/mL, about 11.8 mg/mL, about 11.9 mg/mL, about 12 mg/mL, about 13 mg/mL, about 14 mg/mL, about 15 mg/mL, about 16 mg/mL, about 17 mg/mL, about 18 mg/mL, about 18.4 mg/mL, about 18.5 mg/mL, about 18.6 mg/mL, about 18.7 mg/mL, about 18.8 mg/mL, about 18.8 mg/mL, about 18.9 mg/mL, about 19 mg/mL, about 10 mg/mL, about 20 mg/mL, about 22.5 mg/mL, about 22.6 mg/mL, about 22.7 mg/mL, about 22.7 mg/mL, about 22.8 mg/mL, about 22.9 mg/mL, about 23 mg/mL, about 23.1 mg/mL, about 23.2 mg/mL, about 23.3 mg/mL, about 23.4 mg/mL, about 23.5 mg/mL, about 30 mg/mL, about 37.1 mg/mL, about 37.2 mg/mL, about 37.3 mg/mL, about 37.4 mg/mL, about 37.5 mg/mL, about 37.6 mg/mL, about 37.7 mg/mL, about 37.8 mg/mL, about 37.9 mg/mL, about 40 mg/mL, about 46.0 mg/mL, about 46.1 mg/mL, about 46.2 mg/mL, about 46.3 mg/mL, about 46.4 mg/mL, about 46.5 mg/mL, about 50 mg/mL, about 55.5 mg/mL, about 55.6 mg/mL, about 55.7 mg/mL, about 55.8 mg/mL, about 55.9 mg/mL, about 56.0 mg/mL, about 68.9 mg/mL, about 69.0 mg/mL, about 69.1 mg/mL, about 69.2 mg/mL, about 69.3 mg/mL, about 69.4 mg/mL, about 69.5 mg/mL, about 74.3 mg/mL, about 74.4 mg/mL, about 74.5 mg/mL, about 74.6 mg/mL, about 74.7 mg/mL, about 74.8 mg/mL, about 74.9 mg/mL, about 75 mg/mL, about 90 mg/mL, about 91.8 mg/mL, about 91.9 mg/mL, about 92.0 mg/mL, about 92.1 mg/mL, about 92.2 mg/mL, about 92.3 mg/mL, about 92.4 mg/mL, about 92.5 mg/mL, about 92.6 mg/mL, about 100 mg/mL, about 111.4 mg/mL, about 111.5 mg/mL, about 111.6 mg/mL, about 111.7 mg/mL, about 111.8 mg/mL, about 111.9 mg/mL, about 112 mg/mL, about 130 mg/mL, about 137.8 mg/mL, about 137.9.0 mg/mL, about 138.0 mg/mL, about 138.1 mg/mL, about 138.2 mg/mL, about 138.3 mg/mL, about 138.4 mg/mL, about 138.5 mg/mL, about 140 mg/mL, about 150 mg/mL, 200 mg/mL, about 250 mg/mL, about 300 mg/mL, about 350 mg/mL, about 400 mg/mL, about 450 mg/mL, about 500 mg/mL.

Some embodiments provide a method for treatment of CNS disorders in a human, including schizophrenia and depressive episodes associated with bipolar disorder, by orally administering to a human subject from about 5 mg/mL to about 200 mg/mL of the stable liquid pharmaceutical composition of the invention.

In some embodiments, the present invention provides oral liquid pharmaceutical composition comprising lurasidone or its pharmaceutically acceptable salts thereof at a concentration of about 1 mg/mL to about 100 mg/mL, at least one pharmaceutically acceptable liquid vehicle; and one or more pharmaceutically acceptable excipients selected from suspending agents, anti-caking agents, bitter blockers, wetting agents, antifoaming agents, taste masking agents, solubilizing agents, salt forming agents, pH adjusting agents, pH modifiers, buffering agents, thickening agents, stabilizers, anti-oxidants, chelating agents, preservatives, flavoring agents, sweetening agents, coloring agents, or mixtures thereof.

The terms “liquid vehicle” or “pharmaceutically acceptable liquid vehicle” or “solvent” or “pharmaceutically acceptable solvent” as used herein, is any liquid medium used for dilution or dissolution of parenteral, oral or peroral formulations, such as water, aqueous organic solvent, non-aqueous organic solvent and other liquids described herein or used in the pharmaceutical and/or food industry. The liquid vehicle of the present invention is selected from water, ringer's solution, isotonic sodium chloride solution, glycerin, alcohols, ethanol, propylene glycol, polyethylene glycol, or mixtures thereof. Preferably water is used as a liquid vehicle. In a preferred embodiment, the inventive composition comprises at least about 50% water, at least about 60% water, at least about 70% water, at least about 80% water, at least about 90% water, preferably at least about 95% water and more preferably at least about 99% water with respect to total amount of the composition.

In another embodiment, the present invention provides stable oral liquid pharmaceutical compositions suitable for oral administration comprising: (i) lurasidone or its pharmaceutically acceptable salts thereof at a concentration of about 1 mg/mL to about 200 mg/mL; (ii) at least one pharmaceutically acceptable liquid vehicle; and (iii) optionally, one or more pharmaceutically acceptable excipients selected from stabilizers, solubilizing agents, salt forming agents, suspending agents, pH modifiers, pH adjusting agents, buffering agents, thickening agents, anti-oxidants, chelating agents, preservatives, flavoring agents, sweetening agents, coloring agents, or mixtures thereof.

In some embodiments, an oral liquid pharmaceutical composition of lurasidone is a solution or a suspension.

In another embodiment, the present invention relates to an oral solution comprising lurasidone or its pharmaceutically acceptable salts thereof, at a concentration of about 1 mg/mL to about 200 mg/mL; at least a solubilizing agent; at least a salt forming agent; at least a suspending agent; at least a buffering agent, at least a flavoring agent, at least a sweetening agent and at least one pharmaceutically acceptable liquid vehicle; wherein the solution is stable when stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 6 months.

In another embodiment, the present invention relates to an oral liquid pharmaceutical composition comprising lurasidone or its pharmaceutically acceptable salts thereof, at a concentration of about 1 mg/mL to about 200 mg/mL; and at least one pharmaceutically acceptable liquid vehicle, wherein the composition is in the form of a solution suitable for oral administration, wherein the solution remains stable when stored at 25° C.±2° C./60% a 5% RH or 40° C.±2° C./75%±5% RH or 30° C.±2° C./65%±5% RH or 40° C.±2° C./25%±5% RH for at least 3 months or at 2-8° C. for at least 6 months.

In another embodiment, the present invention relates to an oral liquid pharmaceutical composition comprising lurasidone or its pharmaceutically acceptable salts thereof, at a concentration of about 1 mg/mL to about 200 mg/mL; and at least one pharmaceutically acceptable liquid vehicle, wherein the composition is in the form of a suspension suitable for oral administration, wherein the suspension remains stable when stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 6 months.

In another embodiment, the oral liquid compositions of the invention are stable for at least 6 months when stored at 25° C.±2° C./60%±5% RH or 40° C.±2° C./75%±5% RH or 30° C.±2° C./65%±5% RH or 40° C.±2° C./25%±5% RH such that the level of impurities in the solution are less than 0.5% w/w as measured by HPLC.

In another embodiment, the present invention relates to stable oral suspension comprising lurasidone or its pharmaceutically acceptable salts thereof; wherein the particle size range of the lurasidone or pharmaceutically acceptable salt thereof in the suspension is in the range of about 0.1 to about 150 microns.

In another embodiment, the present invention relates to stable oral suspension comprising lurasidone or its pharmaceutically acceptable salts thereof, at least one salt forming agent, wherein the ratio of lurasidone to salt forming agent in the composition is in the ratio of about 1:1 to about 1:5, preferably about 1:2.

The pharmaceutical compositions of the present invention contain salt forming agents. The term “salt forming agent” as used herein, is an agent used to facilitate the formation of salts within a pharmaceutical formulation, usually to improve the solubility, bioavailability, or stability of drugs. Such compounds include, by way of example and without limitation, tartaric acid, fumaric acid, succinic acid, acetic acid, phosphoric acid, maleic acid, malic acid, sodium pamoate or salts thereof and others known to those of ordinary skill in the art.

In some embodiments, the inventive oral liquid compositions of lurasidone comprises at least one solubilizing agent. The term “solubilizing agent” as used herein, is an agent used to facilitate solubilizing agents are substances that enhance the solubility of poorly water-soluble drugs, thereby improving their bioavailability. Many active pharmaceutical ingredients (APIs) have limited solubility in water, which can reduce their effectiveness when taken orally or administered in other forms. Solubilizing agents help to overcome this limitation, making drugs more readily absorbed by the body. Such solubilizing agents include, by way of example and without limitation, isopropyl alcohol, methanol, lecithin, mono- and diglycerides, propylene glycol, glycerin, dimethyl sulfoxide (DMSO), polysorbate 80, cyclodextrin, benzyl alcohol, N,N-dimethylacetamide, lactic acid, and others known to those of ordinary skill in the art. In some embodiments, the concentration of solubilizing agent in the present invention ranges from 0.001% w/v to 10% w/v, preferably 0.01% w/v to 5.0% w/v, more preferably 0.05% w/v to 1.0% w/v, based on the total weight of the composition. In some embodiments, the amount of solubilizing agent in the present invention ranges from 1 mg/mL to 100 mg/mL, preferably from about 1 mg/mL to about 50 mg/mL, more preferably from about 1 mg/mL to about 30 mg/mL, more preferably from about 10 mg/mL to about 30 mg/mL, based on the total weight of the composition.

In some embodiments, the inventive oral liquid compositions of lurasidone comprises at least one suspending agent. The term “suspending agent” as used herein, is an agent used to stabilize solid particles (e.g., drugs or excipients) that are dispersed in a liquid medium. These agents prevent the particles from settling at the bottom of the container, ensuring uniform distribution and proper dosing upon administration. The suspending agent improves the physical stability of the suspension, maintaining a consistent particle size and preventing clumping or aggregation. Such “suspending agent” include, by way of example and without limitation, xanthan gum, guar gum, acacia gum, gellan gum, hypromellose (e.g., Methocel® E15), carboxymethylcellulose sodium and hydroxy ethylcellulose, microcrystalline cellulose (Avicel®), a blend of microcrystalline cellulose and carboxymethylcellulose sodium (Avicel® RC 591), sodium alginate, methylcellulose, colloidal silica, and others known to those of ordinary skill in the art. In some embodiments, the concentration of suspending agents in the present invention ranges from 0.001% to 10%, preferably 0.01% to 5.0%, more preferably 0.05% to 2.0%, based on the total weight of the composition.

In some embodiments, the inventive oral liquid compositions of lurasidone further comprises at least a buffering agent. The term “buffer” or “buffering agent” as used herein, is an agent used to resist change in pH upon dilution or addition of acid or alkali. Such “buffer” or “buffering agents” include, by way of example and without limitation, sodium dihydrogen phosphate monohydrate, disodium hydrogen phosphate anhydrous, citric acid monohydrate, anhydrous citric acid, sodium succinate, ascorbic acid, acetic acid, sodium acetate, adipic acid, benzoic acid, sodium benzoate, trisodium citrate, monobasic sodium phosphate, dibasic sodium phosphate, disodium hydrogen phosphate dodecahydrate, lactic acid, tris buffer, tartaric acid, potassium metaphosphate, potassium phosphate, monobasic sodium acetate, sodium ascorbate anhydrous, sodium ascorbate monohydrate, sodium tartrate and others known to those of ordinary skill in the art. In some embodiments, the concentration of buffer in the composition ranges from 0.001% w/v to 10% w/v, preferably 0.01% w/v to 5.0% w/v, more preferably 0.05% w/v to 1.0% w/v, based on the total weight of the composition.

In some embodiments, the concentration of buffer in the composition ranges from about 0.001% w/v to about 10% w/v, based on the total weight of the composition. The buffer concentration may be selected from any sub-range or individual value therein, including concentrations ranging from about 0.001% w/v to about 8% w/v, from about 0.001% w/v to about 5% w/v, from about 0.005% w/v to about 3% w/v, from about 0.01% w/v to about 2% w/v, from about 0.01% w/v to about 1.5% w/v, from about 0.05% w/v to about 1% w/v, or from about 0.1% w/v to about 0.75% w/v. In some embodiments, the buffer concentration is at least about 0.001% w/v, 0.01% w/v, 0.05% w/v, 0.1% w/v, or 0.25% w/v, and is no greater than about 10% w/v, 8% w/v, 5% w/v, 2% w/v, or 1% w/v. In certain embodiments, the buffer concentration is selected from about 0.001% w/v, 0.005% w/v, 0.01% w/v, 0.05% w/v, and from about 0.1% w/v to about 10% w/v in increments of about 0.1% w/v, including about 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, 2.1% w/v, 2.2% w/v, 2.3% w/v, 2.4% w/v, 2.5% w/v, 2.6% w/v, 2.7% w/v, 2.8% w/v, 2.9% w/v, 3.0% w/v, 3.1% w/v, 3.2% w/v, 3.3% w/v, 3.4% w/v, 3.5% w/v, 3.6% w/v, 3.7% w/v, 3.8% w/v, 3.9% w/v, 4.0% w/v, 4.1% w/v, 4.2% w/v, 4.3% w/v, 4.4% w/v, 4.5% w/v, 4.6% w/v, 4.7% w/v, 4.8% w/v, 4.9% w/v, 5.0% w/v, 5.1% w/v, 5.2% w/v, 5.3% w/v, 5.4% w/v, 5.5% w/v, 5.6% w/v, 5.7% w/v, 5.8% w/v, 5.9% w/v, 6.0% w/v, 6.1% w/v, 6.2% w/v, 6.3% w/v, 6.4% w/v, 6.5% w/v, 6.6% w/v, 6.7% w/v, 6.8% w/v, 6.9% w/v, 7.0% w/v, 7.1% w/v, 7.2% w/v, 7.3% w/v, 7.4% w/v, 7.5% w/v, 7.6% w/v, 7.7% w/v, 7.8% w/v, 7.9% w/v, 8.0% w/v, 8.1% w/v, 8.2% w/v, 8.3% w/v, 8.4% w/v, 8.5% w/v, 8.6% w/v, 8.7% w/v, 8.8% w/v, 8.9% w/v, 9.0% w/v, 9.1% w/v, 9.2% w/v, 9.3% w/v, 9.4% w/v, 9.5% w/v, 9.6% w/v, 9.7% w/v, 9.8% w/v, 9.9% w/v, and 10.0% w/v, including any intermediate value or combination of values falling within the disclosed range. The buffer concentration may be chosen based on factors including the nature of the composition, desired pH range, storage conditions, compatibility with other components, and intended use, and such selection can be readily performed by a person of ordinary skill in the art without undue experimentation while remaining within the disclosed ranges.

In some embodiments, the inventive oral liquid compositions of lurasidone exhibit resistance to pH change such that the composition has a pH that remains within a range of 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In some embodiments, the inventive oral liquid compositions of lurasidone exhibit resistance to pH change such that the composition has a pH that remains within a range of from about 2.0 to about 6.0, about 2.0 to about 5.0, about 2.0 to about 4.0, about 2.0 to about 3.5, about 2.0 to about 3.0, about 2.0 to about 2.5, about 3.0 to about 6.0, about 3.0 to about 5.0, about 3.0 to about 4.0, about 3.0 to about 3.5, about 4.0 to about 6.0, about 4.0 to about 5.0, about 4.0 to about 4.5, when stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 6 months.

In some embodiments, the inventive oral liquid compositions of lurasidone further comprises at least one “stability enhancing agent” or “stabilizer”. The term “stability enhancing agent” or “stabilizer” as used herein inhibits, prevents, slows down, or reduces the degradation of lurasidone. More specifically, stability enhancing agents may comprise amino acids, salts, ethylenediaminetetraacetic acid (EDTA), metal ions, gums, celluloses, cyclodextrins, sugars, sugar alcohols, monosaccharides, disaccharides or polysaccharides or combinations thereof. In some embodiments, concentration of the stabilizer ranges from 0.001% to 20%, preferably 0.01% to 15.0%, more preferably 0.1% to 10.0%, based on the total weight of the composition.

In some embodiments, the inventive oral liquid compositions of lurasidone further comprise at least one preservative. In addition to stabilizing pharmaceutical preparations against chemical and physical degradation, liquid preparations, especially multi-dose preparations, must usually be protected from microbial contamination. In one embodiment, pharmaceutical composition of the present invention comprise a preservative selected from benzoic acid, sodium benzoate, sodium or potassium salts thereof, ethanol, isopropanol, methanol, butyl alcohol, benzalkonium chloride, benzethonium chloride, benzyl alcohol, butylparaben, cetylpyridinium chloride, chlorobutanol, chlorocresol, cresol, dehydroacetic acid, ethylparaben, ethylparaben sodium, methylparaben, methylparaben sodium, phenol, phenylethyl alcohol, phenylmercuric acetate, phenylmercuric nitrate, potassium benzoate, potassium sorbate, propylparaben, propylparaben sodium, sodium dehydroacetate, sodium propionate, sorbic acid, thimerosal, thymol, or combinations thereof. In a preferred embodiment, the concentration of preservative ranges from 0.001% to 10%, preferably 0.01% to 5.0%, more preferably 0.05% to 1.0%, based on the total weight of the composition.

In some embodiments, the inventive oral liquid compositions of lurasidone further comprise at least one pH modifier, selected from trisodium citrate (also known as sodium citrate), alkanolamines such as monoethanolamine, diethanolamine, triethanolamine, monopropanolamine, dipropanolamine, tripropanolamine, 2-amino-2-methyl-1-propanol, 2-amino-2-hydroxymethyl-1,3-propanediol, or mixtures thereof; amino acids such as glycine, alanine, glutamic acid, L-arginine, lysine, L-cysteine, or methionine and/or mixtures thereof; and acidifying agents such as inorganic and organic acids, including phosphoric acid, acetic acid, ascorbic acid, citric acid, tartaric acid, hydrochloric acid, and/or mixtures thereof.

In some embodiments, the pH modifier is selected from the group comprising trisodium citrate or tartrate and mixtures thereof.

Such pH modifiers are present at a weight percentage ranging from about 0.001% to about 20%, preferably 0.01% to 15.0%, more preferably 0.1% to 10.0%, based on the total weight of the composition.

In some embodiments, the pH modifiers are present at an amount ranging from about 0.1 mg/mL to about 10 mg/mL, such as about 0.3 mg/mL to about 8 mg/mL, about 0.5 mg/mL to about 7.5 mg/mL, about 1.5 mg/mL to about 6 mg/mL, or about 5 mg/mL to about 7 mg/mL.

In some embodiments of the present invention, a stable oral solution of lurasidone comprises (i) lurasidone or its pharmaceutically acceptable salts thereof at a concentration of about 1 mg/mL to about 200 mg/mL; (ii) at least one pharmaceutically acceptable liquid vehicle; (iii) one or more other pharmaceutically acceptable excipients, wherein pH of the solution is in the range of 2 to 6; and wherein impurities level of in the solution is less than 0.5% w/w as measured by HPLC, when the solution is stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 12 months.

In some embodiments, the inventive oral liquid compositions further comprise at least one chelating agent. The term “chelating agent” as used herein, refers to an agent which forms stable complexes with metal ions via two or more of its functional groups. Preferably, the chelating agent is selected from the group consisting of disodium ethylenediaminetetraacetic acid (disodium EDTA), diethylenetriaminepentaacetic acid (DTPA), ethylene glycol-bis (β-aminoethyl ether)-tetra acetic acid (EGTA), N-(hydroxyethyl) ethylenediaminetriacetic acid (HEDTA), nitrilotriacetic acid (NTA), triethanolamine, 8-hydroxyquinoline, gluconic acid, saccharic acid, thiodipropionic acid, acetonic dicarboxylic acid, lecithin, di(hydroxyethyl)glycine, phenylalanine, tryptophan, sorbitol and pharmaceutically acceptable salts and mixtures thereof. More preferably, the chelating agent is selected from the group consisting of disodium EDTA, DTPA, gluconic acid and a pharmaceutically acceptable salts and mixtures thereof. The amount of chelating agent in inventive compositions range from about 0.01 mg/mL to about 1 mg/mL of the composition. In some embodiments, the chelating agent concentration ranges from about 0.001% to about 5% w/v of total composition.

In some embodiments, the inventive oral liquid compositions of the present invention further comprises at least one sweetening agent. The term “sweetening agent” as used herein, refers to both bulk (caloric) and intense (non-caloric) sweetening agent, which impart sweet taste to the preparation. Examples of bulk sweetening agents are dextrose, fructose, glucose, hydrogenated glucose syrup, isomalt, maltitol, maltose, mannitol, sorbitol, sucrose, xylitol, ribose, deoxyribose, neuraminic acid and mixtures thereof. Examples of intense sweetening agents are acesulfame, alitame, aspartame, cyclamate, dihydrochalcone sweetener, monellin, neohesperidin, neotame, saccharin, stevioside, sucralose, pharmaceutically acceptable salts thereof, such as sodium or calcium saccharin, acesulfame potassium or sodium cyclamate, and mixtures thereof. In one embodiment, the pharmaceutically acceptable sweetening agent in the present invention is sucralose. In a preferred embodiment, the concentration of sweetening agent ranges from 0.001% to 10%, preferably 0.01% to 5.0%, more preferably 0.05% to 1.0%, based on the total weight of the composition.

In some embodiments, the inventive oral liquid compositions of the present invention further comprises at least one flavoring agent. The term “flavoring agent” as used herein, refers to an agent or a mixture of agents that adds flavor to a mixture. Flavoring agents include natural flavors, artificial flavors, and mixtures thereof. Flavoring agents include, but are not limited to, mint, peppermint, cola, apple, vanilla, orange, peach, apricot, raspberry, cherry, honey, lemon, coconut, pineapple, strawberry banana, mixed berry, mixed red fruit and cream flavors and mixture thereof. In some embodiments, the flavoring agent is mixed berry flavor. The concentration of flavoring agent ranges from 0.001% to 10%, preferably 0.01% to 5.0%, more preferably 0.05% to 1.0%, based on the total weight of the composition.

In some embodiments, the inventive oral liquid compositions of the present invention further comprises at least one thickening agent. The term “thickening agent” as used herein, to thicken the liquid composition and to improve the mouth-feel of the composition, and/or to help coat the lining of the gastrointestinal tract, thickening agents can be optionally included into the present pharmaceutical composition. Preferably, the thickening agent is selected from but not limited to acacia, alginic acid bentonite, carbomer, carboxymethylcellulose calcium or sodium, cetostearyl alcohol, methyl cellulose, ethylcellulose, gelatin guar gum, hydroxyethylcellulose (HEC), hydroxypropylcellulose (HPC), hydroxypropylmethylcellulose (HPMC), maltodextrin, polyvinyl alcohol, povidone, propylene carbonate, propylene glycol alginate, sodium alginate, sodium starch glycolate, starch tragacanth, and xanthan gum, or any combination thereof. In some embodiments, the concentration of the thickening agent ranges from 0.001% to 20%, preferably 0.01% to 15.0%, more preferably 0.1% to 10.0%, based on the total weight of the composition.

In some embodiments, the inventive oral liquid compositions of the present invention further comprises at least one wetting agent. The term “wetting agent” as used herein, refer to substance used to reduce the surface tension between a liquid and a solid, thereby improving the ability of the liquid to spread over or penetrate a solid surface, such as a drug particle. This property is crucial in for improving the dissolution, bioavailability, and stability of poorly soluble drugs. Such “wetting agent” include, by way of example and without limitation, Poloxamer (P188), polyethylene glycols (PEGs), sorbitan monolaurate, polysorbate 80, polysorbate 20, sodium lauryl sulfate, sorbitan esters, lecithin and others known to those of ordinary skill in the art. In some embodiments, the concentration of wetting agent in the present invention ranges from 0.001% to 10%, preferably 0.01% to 5.0%, more preferably 0.05% to 2.0%, based on the total weight of the composition.

In some embodiments, the inventive oral liquid compositions of the present invention further comprises at least one antifoaming agent or a defoamer. The term “antifoaming agent” or “defoamer” as used herein, is a chemical additive that reduces and hinders the formation of foam in the preparation of liquid composition. The terms antifoaming agent and defoamer are often used interchangeably. Commonly used agents are insoluble oils, polydimethylsiloxanes (e.g., simethicone) and other silicones, certain alcohols, stearates and glycols. Simethicone is available as a pure material (100%) and in combination with other excipients to facilitate dispersion and handling. Common simethicone containing products include a 30% w/w simethicone solid sold under the trademark NuSil MED-342, 100% simethicone liquids sold under the trademark NuSil Med-340, Med-346, and Med-347, 30% simethicone emulsions sold under the trademark Dow Corming® Q7-2587, 7-9245, and Medical Antifoam C. The additive is used to prevent formation of foam or is added to break foam already formed. Antifoaming agents reduce foaming in the preparation of liquid formulations which can result in coagulation of aqueous dispersions.

In some embodiments, the lurasidone oral liquid compositions described herein comprises an antifoaming agent. In some embodiments, the antifoaming agent is simethicone.

In some embodiments, the antifoaming agent is present in about 0.05 mg/ml to about 1.0 mg/ml in the liquid formulation. In some embodiments, the antifoaming agent is present in about 0.05 mg/ml to about 2.0 mg/ml in the liquid formulation. In some embodiments, the antifoaming agent is present in about 0.05 mg/ml to about 2.5 mg/ml in the liquid formulation. In other embodiments, the antifoaming agent is present in about 0.05 mg/ml, about 0.1 mg/ml, about 0.15 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.65 mg/ml, about 0.7 mg/ml, about 0.75 mg/ml, about 0.8 mg/ml, about 0.85 mg/ml, about 0.9 mg/ml, about 0.95 mg/ml, about 1.0 mg/ml, about 1.1 mg/ml, about 1.15 mg/ml, about 1.2 mg/ml, about 1.25 mg/ml, about 1.3 mg/ml, about 1.35 mg/ml, about 1.4 mg/ml, about 1.45 mg/ml, about 1.5 mg/ml, about 1.55 mg/ml, about 1.6 mg/ml, about 1.65 mg/ml, about 1.7 mg/ml, about 1.75 mg/ml, about 1.8 mg/ml, about 1.85 mg/ml, about 1.9 mg/ml, about 1.95 mg/ml, about 2.0 mg/ml, about 2.05 mg/ml, about 2.1 mg/ml, about 2.15 mg/ml, about 2.2 mg/ml, about 2.25 mg/ml, about 2.3 mg/ml, about 2.35 mg/ml, about 2.4 mg/ml, about 2.45 mg/ml, or about 2.5 mg/ml in the liquid formulation. In other embodiments, the antifoaming agent is present in about 0.6 mg/ml in the liquid formulation. In other embodiments, the antifoaming agent is present in about 2.0 mg/ml in the liquid formulation. In some embodiments, the antifoaming agent is present in about 0.05 mg/ml to about 0.3 mg/ml in the liquid formulation. In some embodiments, the antifoaming agent is present in about 0.1 mg/ml to about 0.2 mg/ml in the liquid formulation. In some embodiments, the antifoaming agent is simethicone and is present in about 0.15 mg/ml in the liquid formulation.

In some embodiments, the antifoaming agent is present in about 0.1% w/w to about 7% w/w of the solids in the liquid formulation. In other embodiments, the antifoaming agent is present in about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6% w/w, about 6.1% w/w, about 6.2% w/w, about 6.3% w/w, about 6.4% w/w, about 6.5% w/w, about 6.6% w/w, about 6.7% w/w, about 6.8% w/w, about 6.9% w/w, or about 7% w/w of the solids in the liquid formulation.

In some embodiments, the inventive oral liquid compositions of the present invention further comprise at least one surfactants. The term “surfactant” as used herein refers to the compounds that lower the surface tension (or interfacial tension) between two liquids or between a liquid and a solid. Most commonly, surfactants are classified according to polar head group. A non-ionic surfactant has no charged groups in its head. The head of an ionic surfactant carries a net positive, or negative charge. If the charge is negative, the surfactant is more specifically called anionic; if the charge is positive, it is called cationic. If a surfactant contains a head with two oppositely charged groups, it is termed zwitterionic. Anionic surfactants contain anionic functional groups at their head, such as sulfate, sulfonate, phosphate, and carboxylates. Prominent alkyl sulfates include ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), and sodium myreth sulfate. Others include: docusate (dioctyl sodium sulfosuccinate), perfluorooctanesulfonate (PFOS), perfiuorobutanesulfonate, alkyl-aryl ether phosphates, alkyl ether phosphates. Cationic surfactant include pH-dependent primary, secondary, or tertiary amines such as octenidine dihydrochloride; and permanently charged quatermary ammonium salts such as cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB). Zwitterionic (amphoteric) surfactants have both cationic and anionic centers attached to the same molecule. The cationic part is based on primary, secondary, or tertiary amines or quatermary ammonium cations. The anionic part can be more variable and include sulfonates, as in the sultaines CHAPS (3[(3-cholamidopropyl)dimethylammonio]-1-propanesulfonate) and cocamidopropyl hydroxysultaine. Betaines such as cocamidopropyl betaine have a carboxylate with the ammonium. The most common biological zwitterionic surfactants have a phosphate anion with an amine or ammonium, such as the phospholipids phosphatidylserine, phosphatidylethanolamine, phosphatidylcholine, and sphingomyelins. Nonionic surfactants include fatty alcohols, cetyl alcohol, stearyl alcohol, and cetostearyl alcohol, and oleyl alcohol. Also used as nonionic surfactants are polyethylene glycol alkyl ethers (such as octaethylene glycol monododecyl ether, pentaethylene glycol monododecyl ether), polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate), polyoxyethylene glycol sorbitan alkyl esters (such as polysorbate), sorbitan alkyl esters (such as Spans), cocamide MEA, cocamide DEA, dodecyldimethylamine oxide, block copolymers of polyethylene glycol and polypropylene glycol (such as poloxamers), and polyethoxylated tallow amine (POEA). The most commonly used surfactants are fatty acid esters of sorbitan polyethoxylates, i.e. polysorbate 20 and polysorbate 80. The two differ only in the length of the aliphatic chain that imparts hydrophobic character to the molecules, C-12 and C-18, respectively. Polysorbate 80 is more surface-active and has a lower critical micellar concentration than polysorbate 20.

In some embodiments, the lurasidone fumarate liquid formulation described herein comprises a surfactant. In some embodiments, the surfactant is polysorbate 80.

In some embodiments, the surfactant is added in separate portions while preparing the inventive oral liquid pharmaceutical compositions.

In some embodiments, the surfactant is present in about 0.1 mg/ml to about 3.0 mg/ml in the liquid formulation. In other embodiments, the surfactant is present in about 0.1 mg/ml, about 0.15 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.65 mg/ml, about 0.7 mg/ml, about 0.75 mg/ml, about 0.8 mg/ml, about 0.85 mg/ml, about 0.9 mg/ml, about 0.95 mg/ml, about 1.0 mg/ml, about 1.1 mg/ml, about 1.15 mg/ml, about 1.2 mg/ml, about 1.25 mg/ml, about 1.3 mg/ml, about 1.35 mg/ml, about 1.4 mg/ml, about 1.45 mg/ml, about 1.5 mg/ml, about 1.55 mg/ml, about 1.6 mg/ml, about 1.65 mg/ml, about 1.7 mg/ml, about 1.75 mg/ml, about 1.8 mg/ml, about 1.85 mg/ml, about 1.9 mg/ml, about 1.95 mg/ml, about 2.0 mg/ml, about 2.1 mg/ml, about 2.15 mg/ml, about 2.2 mg/ml, about 2.25 mg/ml, about 2.3 mg/ml, about 2.35 mg/ml, about 2.4 mg/ml, about 2.45 mg/ml, about 2.5 mg/ml, about 2.55 mg/ml, about 2.6 mg/ml, about 2.65 mg/ml, about 2.7 mg/ml, about 2.75 mg/ml, about 2.8 mg/ml, about 2.85 mg/ml, about 2.9 mg/ml, about 2.95 mg/ml, or about 3.0 mg/ml in the liquid formulation.

In some embodiments, the surfactant is present in about 1% w/v to about 15% w/v of the solids in the liquid formulation. In other embodiments, the surfactant is present in about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% of the solids in the liquid formulation.

In some embodiments, the surfactant is present in about 1% w/v to about 5% w/v of the solids in the liquid formulation. In other embodiments, the surfactant is present in about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% of the solids in the liquid formulation.

In some embodiments, the inventive oral liquid compositions of the present invention comprise at least one anticaking agent. The term “anticaking agent” as used herein refers to the compounds that improve re-suspendability of the formulation. The anticaking agent is selected from but not limited to colloidal silica and/or colloidal silicon dioxide, calcium phosphate tribasic, magnesium oxide, magnesium silicate, calcium silicate or combinations thereof.

In some embodiments, the anticaking agent is present in about 0.1 mg/ml to about 3.0 mg/ml in the liquid formulation. In other embodiments, the surfactant is present in about 0.1 mg/ml, about 0.15 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.65 mg/ml, about 0.7 mg/ml, about 0.75 mg/ml, about 0.8 mg/ml, about 0.85 mg/ml, about 0.9 mg/ml, about 0.95 mg/ml, about 1.0 mg/ml, about 1.1 mg/ml, about 1.15 mg/ml, about 1.2 mg/ml, about 1.25 mg/ml, about 1.3 mg/ml, about 1.35 mg/ml, about 1.4 mg/ml, about 1.45 mg/ml, about 1.5 mg/ml, about 1.55 mg/ml, about 1.6 mg/ml, about 1.65 mg/ml, about 1.7 mg/ml, about 1.75 mg/ml, about 1.8 mg/ml, about 1.85 mg/ml, about 1.9 mg/ml, about 1.95 mg/ml, about 2.0 mg/ml, about 2.1 mg/ml, about 2.15 mg/ml, about 2.2 mg/ml, about 2.25 mg/ml, about 2.3 mg/ml, about 2.35 mg/ml, about 2.4 mg/ml, about 2.45 mg/ml, about 2.5 mg/ml, about 2.55 mg/ml, about 2.6 mg/ml, about 2.65 mg/ml, about 2.7 mg/ml, about 2.75 mg/ml, about 2.8 mg/ml, about 2.85 mg/ml, about 2.9 mg/ml, about 2.95 mg/ml, or about 3.0 mg/ml in the liquid formulation.

In some embodiments, the anticaking is present in about 0.1% w/v to about 5% w/v of the composition. In other embodiments, the anticaking is present in about 0.1%, about 0.2%, about 0.3%, about 0.4%, about 0.5%, about 0.6%, about 0.7%, about 0.8%, about 0.9%, about 0%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, or about 4% w/v of the composition.

A “bitter compound” refers to a lurasidone HCl that elicits a detectable bitter flavor in a subject. The term “bitter receptor blocker” or “bitter blocker” as used herein refers to a substance that reduces or prevents the bitter taste caused by lurasidone HCL. The bitter blocker is selected from but not limited to adenosine 5′-monophosphate, salsery, homoeriodictyol sodium, 3β-hydroxydihydrocostunolide, gingerdione, masking 2521 or a combination thereof.

In some embodiments, the bitter blocker is present in about 0.1 mg/ml to about 10.0 mg/ml in the liquid formulation. In other embodiments, the surfactant is present in about 0.1 mg/ml, about 0.15 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.65 mg/ml, about 0.7 mg/ml, about 0.75 mg/ml, about 0.8 mg/ml, about 0.85 mg/ml, about 0.9 mg/ml, about 0.95 mg/ml, about 1.0 mg/ml, about 1.1 mg/ml, about 1.15 mg/ml, about 1.2 mg/ml, about 1.25 mg/ml, about 1.3 mg/ml, about 1.35 mg/ml, about 1.4 mg/ml, about 1.45 mg/ml, about 1.5 mg/ml, about 1.55 mg/ml, about 1.6 mg/ml, about 1.65 mg/ml, about 1.7 mg/ml, about 1.75 mg/ml, about 1.8 mg/ml, about 1.85 mg/ml, about 1.9 mg/ml, about 1.95 mg/ml, about 2.0 mg/ml, about 2.1 mg/ml, about 2.15 mg/ml, about 2.2 mg/ml, about 2.25 mg/ml, about 2.3 mg/ml, about 2.35 mg/ml, about 2.4 mg/ml, about 2.45 mg/ml, about 2.5 mg/ml, about 2.55 mg/ml, about 2.6 mg/ml, about 2.65 mg/ml, about 2.7 mg/ml, about 2.75 mg/ml, about 2.8 mg/ml, about 2.85 mg/ml, about 2.9 mg/ml, about 2.95 mg/ml, about 3.0, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 4.9 mg/ml, about 5.0 mg/ml, about 5.1 mg/ml, about 5.2 mg/ml, about 5.3 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6.0 mg/ml, about 6.1 mg/ml, about 6.2 mg/ml, about 6.3 mg/ml, or about 6.4 mg/ml, about 6.5 mg/ml, about 6.6 mg/ml, about 6.7 mg/ml, about 6.8 mg/ml, about 6.9 mg/ml, about 7.0 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8.0 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9.0 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, or about 10.0 mg/ml in the liquid formulation.

In some embodiments, the bitter blocker is present in about 0.01% w/v to about 10% w/v of the composition. In other embodiments, the anticaking is present in about 0.01%, about 0.02%, about 0.03%, about 0.04%, about 0.05%, about 0.06%, about 0.07%, about 0.08%, 0.085%, about 0.09%, about 0.1%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, or about 3.0% w/v of the composition.

In some embodiments, the inventive oral liquid compositions of the present invention comprise at least one co-solvent. The term “co-solvent” as used herein refers to the liquid components often used to increase the water solubility of drugs which do not contain ionizable group(s) and whose solubility can thus not be increased by pH adjustment. The co-solvents is selected from but not limited to Labrafac lipophile WL 1349/Sorbitol solution/Castor Oil/Soyabean Oil Glycerol monolinoleate EP/Glyceryl monolinoleate NF, Propylene Glycol, glycerol, the low molecular weight PEGs, Polyethylene glycol 4000 (PEG 400) and the like. Furthermore, the present invention comprises about 0.1% to 10.0% of co-solvents of the total composition.

In some embodiments, the co-solvent is present in about 0.01% w/w to about 10% of the composition. In other embodiments, the co-solvent is present in about 0.01%, about 0.02%, about 0.03%, about 0.04%, about 0.05%, about 0.06%, about 0.07%, about 0.08%, 0.085%, about 0.09%, about 0.1%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3.0%, about 4.0%, about 5.0% about 6.0%, about 7.0%, about 8.0%, about 9.0%, or about 10.0% of the composition.

In some embodiments, the present invention relates to a stable oral liquid pharmaceutical composition comprising lurasidone or its pharmaceutical acceptable salts thereof, and one or more excipients selected from solubilizing agent, salt forming agent, one or more suspending agents, wetting agents, anti-foaming agents, anti-caking agents, buffering agents, bitter blockers, sweetening agents, flavoring agents and vehicle; wherein the pH of the liquid composition is ranging from 1.5 to 5.0.

In some embodiments, the present invention relates to a stable oral liquid pharmaceutical composition comprising:
(a) a therapeutically effective amount of lurasidone or a pharmaceutically acceptable salt thereof,
(b) at least one pharmaceutically acceptable liquid vehicle; and
(c) optionally, one or more excipients selected from group consisting of suspending agents, preservatives, buffering agents, wetting agents, anti-foaming agents, anti-caking agents, bitter blockers, thickening agents, pH modifiers, anti-oxidants, complexing agents, chelating agents, flavoring agents, sweetening agents, coloring agents or combinations thereof;

A stable oral liquid pharmaceutical composition comprising:
(a) lurasidone or a pharmaceutically acceptable salt thereof,
(b) at least one pharmaceutically acceptable liquid vehicle; and
(c) optionally, one or more excipients selected from group consisting of suspending agents, preservatives, buffering agents, wetting agents, anti-foaming agents, anti-caking agents, bitter blockers, thickening agents, pH modifiers, anti-oxidants, complexing agents, chelating agents, flavoring agents, sweetening agents, coloring agents or combinations thereof;
   wherein the pH of the pharmaceutical composition is in a range from about 1.5 to about 5; and
   wherein the pharmaceutical composition is stable for at least 1 month when stored at about 40° C. and about 75% relative humidity (RH).

In some embodiments, the present invention relates to a stable oral liquid pharmaceutical composition comprising:
(a) a therapeutically effective amount of lurasidone or a pharmaceutically acceptable salt thereof,
(b) at least one pharmaceutically acceptable liquid vehicle; and
(c) optionally, one or more excipients selected from group consisting of suspending agents, preservatives, buffering agents, wetting agents, anti-foaming agents, anticaking agents, bitter blockers, thickening agents, pH modifiers, anti-oxidants, complexing agents, chelating agents, flavoring agents, sweetening agents, coloring agents or combinations thereof, wherein the pH of the pharmaceutical composition is in a range of about 2 to about 6; and
   wherein the pharmaceutical composition is stable for at least 1 month when stored at about 40° C. and about 75% relative humidity (RH).

In some embodiments, the present invention relates to a stable oral liquid pharmaceutical composition, wherein the composition is a suspension, a solution or an emulsion.

In some embodiments, the present invention relates to a stable oral liquid pharmaceutical composition, wherein the composition is an aqueous suspension.

In some embodiments, the present invention relates to a stable oral liquid pharmaceutical composition, wherein the pH of the composition is from about 2 to about 5.

In some embodiments, the present invention relates to a stable oral liquid pharmaceutical composition, wherein the pH of the composition is from about 2 to about 4.

In one embodiment, the present invention provides a stable oral liquid pharmaceutical composition having a pH in the range of about 2 to about 3.

In a further embodiment, the composition comprises a buffering agent in a concentration ranging from about 0.1% w/v to about 5% w/v.

In yet another embodiment, the composition further comprises a suspending agent in a concentration ranging from about 0.01% w/v to about 10% w/v.

In some embodiments, the present invention relates to a stable oral liquid pharmaceutical composition, wherein concentration of buffering agent is from 0.1% w/v to about 5% w/v.

In some embodiments, the present invention relates to a stable oral liquid pharmaceutical composition, wherein the concentration of suspending agents are in a range of 0.01% to about 10% w/v.

In some embodiments, the present invention relates to a stable oral liquid pharmaceutical composition, wherein the ratio of lurasidone or a pharmaceutical acceptable salt thereof to the one or more suspending agents is about 1:0.5 to about 1:10.

In some embodiments, the present invention relates to a stable oral liquid pharmaceutical composition, wherein the preservative is in a range of 0.1% to about 5% w/v.

In some embodiments, the present invention relates to a stable oral liquid pharmaceutical composition, wherein the wetting agent is in a range of 0.01% to about 5% w/v.

In some embodiments, the present invention relates to a stable oral liquid pharmaceutical composition, wherein a concentration of anti-caking agent is in a range of 0.01% to about 10% w/v.

In some embodiments, the present invention relates to a stable oral liquid pharmaceutical composition, wherein the sweetening agent is in a range of 0.01% to about 3% w/v.

In some embodiments of the invention, the oral suspension comprising lurasidone further comprises a taste masking agent. Palatability of the oral suspension comprising lurasidone is improved by the addition of a taste masking agent. More preferably the taste masking agent is present in an amount of 5 to 50 mg/mL, more preferably 10-30 mg/mL, and most preferably about 20 mg/mL. Taste masking agents selected from the group consisting of glycerol, kleptose, cyclodextrin, ginger, anise, cinnamon, peppermint, licorice, sucrose, glucose, fructose, mannitol, saccharin, aspartame, sucralose, monk fruit, agave syrup, maple syrup, erythritol, xylitol, yacon syrup, neotame, stevia plant, honey or combinations thereof.

In some embodiments, the present invention relates to a stable oral liquid pharmaceutical composition, wherein the composition is a suspension, a solution or an emulsion.

In some embodiments, the present invention relates to a stable oral suspension comprising:
(a) about 0.01% to about 5% w/v of lurasidone or a pharmaceutically acceptable salt thereof;
(b) a pharmaceutically acceptable vehicle;
(c) one or more suspending agents;
(d) one or more buffering agents and
(e) optionally, at least one pharmaceutically acceptable excipient selected from preservatives, wetting agent, anti-foaming agents, anti-caking agents, bitter blockers, thickening agents, pH modifiers, anti-oxidants, complexing agents, chelating agents, flavoring agents, sweetening agents, coloring agents or combinations thereof.

In some embodiments, the present invention relates to a stable oral suspension comprising:
(a) about 0.01% to about 5% w/v of lurasidone or a pharmaceutically acceptable salt thereof;
(b) a pharmaceutically acceptable vehicle;
(c) one or more suspending agents;
(d) one or more buffering agents and
(e) optionally, at least one pharmaceutically acceptable excipient selected from preservatives, wetting agent, anti-foaming agents, anti-caking agents, bitter blockers, thickening agents, pH modifiers, anti-oxidants, complexing agents, chelating agents, flavoring agents, sweetening agents, coloring agents or combinations thereof; and
wherein the pH of the suspension ranges from about 2 to about 6.

In some embodiments, the present invention relates to a stable oral suspension comprising:
(a) about 0.01% to about 5% w/v of lurasidone or a pharmaceutically acceptable salt thereof;
(b) a pharmaceutically acceptable vehicle;
(c) one or more suspending agents;
(d) one or more buffering agents and
(e) optionally, at least one pharmaceutically acceptable excipient selected from preservatives, wetting agent, anti-foaming agents, anti-caking agents, bitter blockers, thickening agents, pH modifiers, anti-oxidants, complexing agents, chelating agents, flavoring agents, sweetening agents, coloring agents or combinations thereof; and
wherein the suspension is stable for at least 1 month when stored at about 40° C. and about 75% relative humidity (RH).

In some embodiments, the present invention relates to a stable oral suspension comprising:
(a) about 0.01% to about 5% w/v of lurasidone or a pharmaceutically acceptable salt thereof;
(b) at least a pharmaceutically acceptable vehicle;
(c) one or more suspending agents;
(d) one or more buffering agents and
(e) optionally, at least one pharmaceutically acceptable excipient selected from preservatives, wetting agent, anti-foaming agents, anti-caking agents, bitter blockers, thickening agents, pH modifiers, anti-oxidants, complexing agents, chelating agents, flavoring agents, sweetening agents, coloring agents or combinations thereof; and
wherein the suspension is stable for at least 1 month when stored at about 60° C.

In some embodiments, the present invention relates to a stable oral suspension comprising:
(a) about 0.01% to about 5% w/v of lurasidone or a pharmaceutically acceptable salt thereof;
(b) a pharmaceutically acceptable vehicle;
(c) one or more suspending agents;
(d) one or more buffering agents and
(e) optionally, at least one pharmaceutically acceptable excipient selected from preservatives, wetting agent, anti-foaming agents, anti-caking agents, bitter blockers, thickening agents, pH modifiers, anti-oxidants, complexing agents, chelating agents, flavoring agents, sweetening agents, coloring agents or combinations thereof;
wherein the pH of the composition ranges from about 2 to about 6; and
wherein the suspension is stable for at least 1 month when stored at about 40° C. and about 75% relative humidity (RH).

In some embodiments, the present disclosure relates to an oral suspension comprising a therapeutically effective amount of lurasidone or a pharmaceutically acceptable salt thereof and a pharmaceutically acceptable vehicle, wherein the lurasidone used in the preparation of the suspension has a D90 particle size of about 100 μm or less, when measured by a light scattering particle size analyzer.

In some embodiments, the present disclosure relates to an oral suspension comprising a therapeutically effective amount of lurasidone or a pharmaceutically acceptable salt thereof and a pharmaceutically acceptable vehicle, wherein the lurasidone used in the preparation of the suspension has a D90 particle size of about 50 μm or less, when measured by a light scattering particle size analyzer.

In some embodiments, the present disclosure relates to an oral suspension comprising a therapeutically effective amount of lurasidone or a pharmaceutically acceptable salt thereof and a pharmaceutically acceptable vehicle, wherein the lurasidone used in the preparation of the suspension has a D90 particle size of about 25 μm or less, when measured by a light scattering particle size analyzer.

In some embodiments, the present disclosure relates to an oral suspension comprising a therapeutically effective amount of lurasidone or a pharmaceutically acceptable salt thereof and a pharmaceutically acceptable vehicle, wherein the lurasidone used in the preparation of the suspension has a D90 particle size of about 6 μm or less, when measured by a light scattering particle size analyzer.

In some embodiments, the present disclosure relates to an oral suspension comprising a therapeutically effective amount of lurasidone or a pharmaceutically acceptable salt thereof and a pharmaceutically acceptable vehicle, wherein the particles of lurasidone or pharmaceutical salt thereof in the suspension have a D90 equal to or less than about 100 μm, when measured by a light scattering particle size analyzer, and wherein at least about 70 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles of lurasidone or pharmaceutical salt thereof in the suspension have a D90 equal to or less than about 50 μm, when measured by a light scattering particle size analyzer, and wherein at least about 70 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles of lurasidone or pharmaceutical salt thereof in the suspension have a D90 equal to or less than about 6 μm, when measured by a light scattering particle size analyzer, and wherein at least about 70 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 100 μm, when measured by a light scattering particle size analyzer, and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 50 μm, when measured by a light scattering particle size analyzer, and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 25 μm, when measured by a light scattering particle size analyzer, and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 100 μm, as measured by a light scattering particle size analyzer after storage for at least 12 months and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 100 μm, as measured by a light scattering particle size analyzer after storage for at least 6 months and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present disclosure relates to an oral suspension comprising a therapeutically effective amount of lurasidone or a pharmaceutically acceptable salt thereof and a pharmaceutically acceptable vehicle, wherein the particles in the suspension have a D90 equal to or less than about 100 μm, as measured by a light scattering particle size analyzer after storage for at least 3 months and wherein at least about 80 wt % of lurasidone dissolves within about 15 minutes when measured using a USP Apparatus II at a paddle rotation speed of 50 rpm in 900 mL of a dissolution medium at 37±0.5° C., in pH 3.8 McIlvaine buffer dissolution medium.

In some embodiments, the present invention relates to a stable oral suspension, wherein the lurasidone or the pharmaceutically acceptable salt thereof has a particle size of about 1 nanometers to 1000 nanometers.

In some embodiments, the present invention relates to a stable oral suspension comprising lurasidone or the pharmaceutically acceptable salt thereof, wherein the lurasidone has a particle size of from about 1 nanometers (nm) to about 1000 nanometers (nm) as measured by Malvern Mastersizer 3000 (laser diffraction). For example, the stable oral suspension have a particle size distribution with a D90 particle size from about 1 nm to about 900 nm, about 1 nm to about 800 nm, about 1 nm to about 700 rm, about 1 nm to about 600 rm, about 1 nm to about 500 nm, about 1 nm to about 400 nm, about 1 nm to about 300 nm, about 1 nm to about 200 nm, about 1 nm to about 150 nm, about 1 nm to about 100 nm, about 1 nm to about 75 nm, about 1 nm to about 50 rm, about 1 nm to about 25 nm, about 1 nm to about 20 nm, or about 1 nm to about 10 nm. As another example, stable oral suspension comprising lurasidone may have a particle size distribution with a D90 particle size of about 5 nm, about 10 nm, about 15 nm, about 20 nm, about 25 nm, about 50 nm, about 75 nm, about 100 nm, about 150 nm, about 200 nm, about 250 nm, about 300 nm, about 350 nm, about 400 nm, about 450 nm, about 500 nm, 550 nm about 600 nm, about 650 nm, about 700 nm, 750 nm about 800 nm, 850 nm about 900 nm, 950 nm, or about 1000 nm.

In some embodiments, the present invention relates to a stable oral suspension comprising lurasidone or the pharmaceutically acceptable salt thereof, wherein the lurasidone has a D50 particle size of from about 1 nanometers (nm) to about 1000 nanometers (nm) as measured by Malvern Mastersizer 3000 (laser diffraction). For example, the stable oral suspension have a particle size distribution with a D50 particle size from about 1 nm to about 900 nm, about 1 nm to about 800 rm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 150 nm, about 1 nm to about 100 nm, about 1 nm to about 75 rm, about 1 nm to about 50 nm, about 1 nm to about 25 nm, about 1 nm to about 20 nm, or about 1 nm to about 10 nm. As another example, stable oral suspension comprising lurasidone may have a particle size distribution with a D50 particle size of about 5 nm, about 10 nm, about 15 nm, about 20 nm, about 25 nm, about 50 nm, about 75 nm, about 100 nm, about 150 nm, about 200 nm, about 250 nm, about 300 nm, about 350 nm, about 400 nm, about 450 rm, about 500 nm, 550 nm about 600 nm, about 650 nm, about 700 nm, 750 nm about 800 nm, 850 nm about 900 nm, 950 nm, or about 1000 nm.

In some embodiments, the present invention relates to a stable oral suspension comprising lurasidone or the pharmaceutically acceptable salt thereof, wherein the lurasidone has a D10 particle size of from about 1 nanometers (nm) to about 1000 nanometers (nm) as measured by Malvern Mastersizer 3000 (laser diffraction). For example, the stable oral suspension have a particle size distribution with a D10 particle size from about 1 nm to about 900 nm, about 1 nm to about 800 rm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 150 nm, about 1 nm to about 100 nm, about 1 nm to about 75 rm, about 1 nm to about 50 nm, about 1 nm to about 25 nm, about 1 nm to about 20 nm, or about 1 nm to about 10 nm. As another example, stable oral suspension comprising lurasidone may have a particle size distribution with a D10 particle size of about 5 nm, about 10 nm, about 15 nm, about 20 nm, about 25 nm, about 50 nm, about 75 nm, about 100 nm, about 150 nm, about 200 nm, about 250 nm, about 300 nm, about 350 nm, about 400 nm, about 450 nm, about 500 nm, 550 nm about 600 nm, about 650 nm about 700 nm, 750 nm about 800 nm, 850 nm about 900 nm, 950 nm, or about 1000 nm.

In some embodiments, the present invention relates to a stable oral suspension, wherein the lurasidone or the pharmaceutically acceptable salt thereof has a particle size of about 0.1 microns (μm) to about 100 microns (μm).

In some embodiments, the present invention relates to a stable oral suspension comprising lurasidone or the pharmaceutically acceptable salt thereof, wherein the lurasidone has a D90 particle size of from about 0.1 μm to about 100 μm as measured by laser diffraction. For example, the stable oral suspension have a particle size distribution with a D90 particle size from about 0.1 μm to about 90 μm, about 0.1 μm to about 80 μm, about 0.1 μm to about 70 μm, about 0.1 μm to about 60 μm, about 0.1 μm to about 50 μm, about 0.1 μm to about 40 μm, about 0.1 μm to about 30 μm, about 0.1 μm to about 20 μm, about 0.1 μm to about 15 μm, about 0.1 μm to about 10 μm, about 0.1 μm to about 9 μm, about 0.1 μm to about 8 μm, about 0.1 μm to about 7 μm, about 0.1 μm to about 6 μm, about 0.1 μm to about 5 μm, about 0.1 μm to about 4 μm, about 0.1 μm to about 3 μm, about 0.1 μm to about 2 μm, or about 0.1 μm to about 1 μm. As another example, stable oral suspension comprising lurasidone may have a particle size distribution with a D90 particle size of about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, 55 μm, about 60 μm, about 75 μm, about 80 μm, about 85 μm, about 90 μm, about 95 μm, or about 100 μm.

In some embodiments, the present invention relates to a stable oral suspension comprising lurasidone hydrochloride, wherein the lurasidone hydrochloride has a D90 particle size of from about 0.1 μm to about 100 μm as measured by laser diffraction. For example, the stable oral suspension have a particle size distribution with a D90 particle size from about 0.1 μm to about 90 μm, about 0.1 μm to about 80 μm, about 0.1 μm to about 70 μm, about 0.1 μm to about 60 μm, about 0.1 μm to about 50 μm, about 0.1 μm to about 40 μm, about 0.1 μm to about 30 μm, about 0.1 μm to about 20 μm, about 0.1 μm to about 15 μm, about 0.1 μm to about 10 μm, about 0.1 μm to about 9 μm, about 0.1 μm to about 8 μm, about 0.1 μm to about 7 μm, about 0.1 μm to about 6 μm, about 0.1 μm to about 5 μm, about 0.1 μm to about 4 μm, about 0.1 μm to about 3 μm, about 0.1 μm to about 2 μm, or about 0.1 μm to about 1 μm. In another example, a stable oral suspension comprising lurasidone hydrochloride may have a particle size distribution with a D90 particle size of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or about 100 μm.

In some embodiments, the present invention relates to a stable oral suspension comprising lurasidone or the pharmaceutically acceptable salt thereof, wherein the lurasidone has a D50 particle size of from about 0.1 μm to about 50 μm as measured by Malvern Mastersizer 3000 (laser diffraction). For example, the stable oral suspension have a particle size distribution with a D50 particle size from about 0.1 μm to about 45 μm, about 0.1 μm to about 40 μm, about 0.1 μm to about 35 μm, about 0.1 nm to about 30 μm, about 0.1 μm to about 25 μm, about 0.1 μm to about 20 μm, about 0.1 μm to about 15 μm, about 0.1 μm to about 10 μm, about 0.1 μm to about 9 μm, about 0.1 μm to about 8 μm, about 0.1 μm to about 7 μm, about 0.1 μm to about 6 μm, about 0.1 μm to about 5 μm, about 0.1 μm to about 4 μm, about 0.1 μm to about 3 μm, about 0.1 μm to about 2 μm, or about 0.1 μm to about 1 μm. As another example, stable oral suspension comprising lurasidone may have a particle size distribution with a D50 particle size of about 0.1 μm, about 0.5 μm, about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, or about 50 μm.

In some embodiments, the present invention relates to a stable oral suspension comprising lurasidone or the pharmaceutically acceptable salt thereof, wherein the lurasidone has a D10 particle size of from about 0.1 μm to about 50 μm as measured by Malvern Mastersizer 3000 (laser diffraction). For example, the stable oral suspension have a particle size distribution with a D10 particle size from about 0.1 μm to about 45 μm, about 0.1 μm to about 40 μm, about 0.1 μm to about 35 μm, about 0.1 μm to about 30 μm, about 0.1 μm to about 25 μm, about 0.1 μm to about 20 μm, about 0.1 μm to about 15 μm, about 0.1 μm to about 10 μm, about 0.1 μm to about 9 μm, about 0.1 μm to about 8 μm, about 0.1 μm to about 7 μm, about 0.1 μm to about 6 μm, about 0.1 μm to about 5 μm, about 0.1 μm to about 4 μm, about 0.1 μm to about 3 μm, about 0.1 μm to about 2 μm, or about 0.1 μm to about 1 μm. As another example, stable oral suspension comprising lurasidone may have a particle size distribution with a D10 particle size of about 0.1 μm, about 0.2 μm, about 0.3 μm, about 0.4 μm, about 0.5 μm, about 0.6 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, or about 50 μm.

In some embodiments, the present invention relates to a stable oral suspension, wherein the composition is a ready-to-use composition that does not need further reconstitution prior to administration.

In some embodiments, the present invention relates to a stable oral suspension, wherein the composition comprises lurasidone or a pharmaceutical acceptable salt thereof at a concentration in a range of about 0.05 mg/mL to about 20 mg/mL.

In another embodiment, the present invention relates to a stable oral suspension, wherein the oral suspension comprises lurasidone or a pharmaceutical acceptable salt thereof at a concentration of about 10 mg/mL.

In some embodiments, the present invention relates to a stable oral suspension comprising
(a) therapeutically effective amount of lurasidone or pharmaceutically acceptable salt thereof;
(b) one or more suspending agents;
(c) one or more buffering agent;
(d) at least one pharmaceutically acceptable vehicle; and
(e) optionally, at least one pharmaceutically acceptable excipient selected from preservatives, wetting agents, anti-foaming agents, anti-caking agents, bitter blockers, thickening agents, pH modifiers, stabilizers, anti-oxidants, chelating agents, flavoring agents, sweetening agents, coloring agents, or combination thereof, and
wherein, as measured using a USP Apparatus II at a paddle rotation speed of 50 rpm in 900 mL of a dissolution medium at 37±0.5° C., the liquid composition exhibits more than about 70 wt % of lurasidone dissolves within about 15 minutes in the dissolution medium, and the dissolution medium is pH 3.8 McIlvaine buffer.

In some embodiments, the present invention relates to a stable oral suspension, wherein the method for treating schizophrenia and/or bipolar depression in a patient in need thereof, comprising administering to a patient in need thereof an oral suspension comprising:
(a) therapeutically effective amount of lurasidone or a pharmaceutically acceptable salt thereof;
(b) at least one suspending agent;
(c) at least one buffering agent;
(d) at least one pharmaceutically acceptable vehicle; and
(e) optionally one or more excipients selected from group consisting of preservatives, buffering agents, wetting agents, anti-foaming agents, anticaking agents, bitter blockers, thickening agents, pH modifiers, stabilizers, anti-oxidants, chelating agents, flavoring agents, sweetening agents, coloring agents or combinations thereof;
wherein the pH of the oral composition ranges from about 2.0 to about 6.0; and
wherein the oral composition is stable after being stored at about 45° C. and about 75% RH for at least 1 month.

In another embodiment, the present disclosure relates to a stable oral suspension comprising:
(a) about 0.1% to about 10% w/v of lurasidone or a pharmaceutically acceptable salt thereof,
(b) about 0.1% to about 5% w/v of one or more suspending agents,
(c) about 0.1% to about 3% w/v of one or more preservatives,
(d) about 0% to about 2% w/v of a wetting agent,
(e) about 0% to about 2% w/v of a anticaking agent,
(f) about 0% to about 1% w/v of an anti-foaming agent,
(g) about 0% to about 1% w/v of one or more buffering agents,
(h) about 0% to about 1% w/v of a sweetening agent,
(i) about 0% to about 1% w/v of a bitter blocker,
(j) about 0% to about 1% w/v of a flavoring agent and
(k) a vehicle;
   wherein the pH of the liquid composition is in the range of about 2.0 to about 6.0; and
   wherein the lurasidone or its pharmaceutically acceptable salt is the only active ingredient in the suspension.

In another embodiment, the present disclosure relates to a stable oral suspension comprising:
(a) about 0.1% to about 10% w/v of lurasidone or a pharmaceutically acceptable salt thereof,
(b) about 0.01% to about 10% w/v of at least one suspending agent;
(c) a pharmaceutically acceptable vehicle; and
   optionally at least one pharmaceutically acceptable excipient selected from preservatives, buffering agents, wetting agents, antifoaming agents, bitter blockers, anticaking agents complexing agents, sweetening agents, flavoring agents, antioxidants or combinations thereof;
   wherein the pH of the suspension ranges from about 2 to about 6 and
   wherein the molar ratio of lurasidone or its pharmaceutically acceptable salt to one or more suspending agents is the range of about 1:0.5 to about 1:10.

In one embodiment, the present disclosure relates to a stable oral suspension comprising;
(a) about 0.1% to about 10% w/v of lurasidone or a pharmaceutically acceptable salt thereof,
(b) about 0.01% to about 10% w/v of at least one suspending agent;
(c) about 0.1% to about 5% w/v of at least one preservative;
(d) a pharmaceutically acceptable vehicle; and optionally at least one pharmaceutically acceptable excipient selected from wetting agents, buffering agents, bitter blockers, antifoaming agents, anticaking agents, complexing agents, sweetening agents, flavoring agents, antioxidants or combinations thereof,
wherein the pH of the suspension ranges from about 2 to about 6 and
wherein the lurasidone used in the preparation of the suspension has a D90 particle size of about 100 μm or less, when measured by a Malvern Mastersizer particle size analyzer.

In one embodiment, the present disclosure relates to a stable oral suspension comprising;
(a) about 0.1% to about 10% w/v of lurasidone or a pharmaceutically acceptable salt thereof,
(b) about 0.01% to about 10% w/v of at least one suspending agent;
(c) about 0.1% to about 5% w/v of at least one preservative;
(d) a pharmaceutically acceptable vehicle; and optionally at least one pharmaceutically acceptable excipient selected from wetting agents, buffering agents, bitter blockers, antifoaming agents, anticaking agents complexing agents, sweetening agents, flavoring agents, antioxidants or combinations thereof,
wherein the pH of the suspension ranges from about 2 to about 6 and
wherein the lurasidone used in the preparation of the suspension has a D50 particle size of about 100 μm or less, when measured by a Malvern Mastersizer particle size analyzer.

In one embodiment, the present disclosure relates to a stable oral suspension comprising;
(a) about 0.1% to about 10% w/v of lurasidone or a pharmaceutically acceptable salt thereof,
(b) about 0.01% to about 10% w/v of at least one suspending agent;
(c) about 0.1% to about 5% w/v of at least one preservative;
(d) a pharmaceutically acceptable vehicle; and
optionally at least one pharmaceutically acceptable excipient selected from wetting agents, buffering agents, bitter blockers, antifoaming agents, anticaking agents complexing agents, sweetening agents, flavoring agents, antioxidants or combinations thereof,
   wherein the pH of the suspension ranges from about 2 to about 6 and
   wherein the lurasidone used in the preparation of the suspension has a D10 particle size of about 100 μm or less, when measured by a Malvern Mastersizer particle size analyzer.

In one embodiment, the present disclosure relates to a stable oral suspension comprising:
(a) about 0.1% to about 10% w/v of lurasidone or a pharmaceutically acceptable salt thereof;
(b) about 0.01% to about 5% w/v of suspending agents selected from xanthan gum, hydroxypropyl methyl cellulose, hydroxyethyl cellulose, microcrystalline cellulose, sodium carboxymethyl cellulose, or a combination thereof;
(c) about 0% to about 5% w/v of colloidal silicone dioxide;
(d) about 0% to about 4% w/v of buffering agents selected from citric acid monohydrate, anhydrous citric acid, disodium hydrogen phosphate sodium citrate, sodium acetate, sodium phosphate, potassium phosphate, tris, sodium tartrate, ascorbic acid or a combination thereof;
(e) about 0% to about 2% w/v of poloxamer;
(g) about 0% to about 1% w/v of simethicone 30% emulsion
(h) about 0% to about 1% w/v of preservative selected from methyl paraben, propyl paraben, sodium propionate, benzyl alcohol, potassium sorbate, sodium benzoate, butylated hydroxyanisole, butylated hydroxytoluene, phenyl ethyl alcohol or a combination thereof;
(i) about 0% to about 1% w/v of sucralose;
(j) about 0% to about 1% w/v of bitter blocker;
(j) about 0% to about 2% w/v of flavoring agent and
(k) a pharmaceutically acceptable vehicle; wherein the pH is in the range of about 2 to about 6.

In some embodiments, the present invention relates to a stable oral suspension, wherein the process comprises:
(i) adding benzyl alcohol to 50% of purified water in a vessel to form a solution;
(ii) adding citric acid monohydrate, disodium hydrogen phosphate dihydrate, poloxamer, and hypromellose sequentially to the solution from (i) to form a solution;
(iii) adding simethicone 30% emulsion to the solution from (ii) to form a homogenous suspension;
(iv) adding colloidal silicon dioxide and lurasidone HCl sequentially to the homogenous suspension from (iii) to form a homogenous suspension;
(v) adding xanthan gum to about 30% of purified water of total quantity in a separate vessel to form a solution;
(vi) adding the solution from step (v) to the homogenous suspension of step (iv) with stirring to form a homogenous suspension;
(vii) adding sucralose, bitter blocker and mixed berry flavor sequentially to the homogenous suspension from (vi), and further with addition of water to form a homogenous suspension; and
(viii) adjusting the pH of the homogenous suspension from (vii) in the range of about 2.0 to 3.0.

In some embodiments, the present invention relates to a stable oral suspension comprising lurasidone or a pharmaceutically acceptable salt thereof, wherein the pH of the composition is from about 2 to about 5.

In some embodiments, the present invention relates to a stable oral suspension comprising lurasidone or a pharmaceutically acceptable salt thereof, wherein the pH of the composition is from about 2 to about 4.

In some embodiments, the present invention relates to a stable oral suspension comprising lurasidone or a pharmaceutically acceptable salt thereof, wherein the pH of the composition is from about 2 to about 3.5.

In some embodiments, the present invention relates to a stable oral suspension comprising lurasidone or a pharmaceutically acceptable salt thereof, wherein the pH of the composition is from about 2 to about 3.

In some embodiments, the present invention relates to a method for treating schizophrenia and/or bipolar depression in a patient in need thereof, comprising administering to a patient in need thereof an oral suspension comprising a dose of lurasidone or a pharmaceutical acceptable salt thereof equivalent to about 9 mg or about 120 mg of lurasidone free base.

In some embodiments, the present invention relates to a stable oral suspension comprising lurasidone or a pharmaceutically acceptable salt thereof, wherein the therapeutically effective amount of lurasidone is equivalent to 5 mg, 9 mg, 9.1 mg, 9.2 mg 9.3 mg, 9.4 mg, 9.5 mg, 9.6 mg 9.7 mg, 9.8 mg, 9.9 mg, 10 mg, 15 mg, 18.6 mg, 19 mg, 20 mg, 27 mg, 27.6 mg, 27.7 mg, 27.8 mg, 27.9 mg, 28 mg, 30 mg, 35 mg, 37 mg, 37.1 mg, 37.25 mg, 37.3 mg, 37.4 mg, 37.5 mg, 39 mg, 40 mg, 45 mg, 50 mg, 55 mg or 60 mg of the lurasidone free base.

In some embodiments, the present invention relates to a stable oral suspension comprising lurasidone or a pharmaceutically acceptable salt thereof, wherein the therapeutically effective amount of lurasidone is equivalent to 5 mg, 9 mg, 9.1 mg, 9.2 mg 9.3 mg, 9.4 mg, 9.5 mg, 9.6 mg 9.7 mg, 9.8 mg, 9.9 mg, 18.6 mg, 27.9 mg, 28 mg, 30 mg, 37.25 mg, 37.3 mg, 37.4 mg, 37.5 mg, 39 mg, 40 mg, 45 mg, 50 mg, 55 mg or 60 mg of the lurasidone free base.

In one embodiment, a pharmaceutical suspension is prepared by first dissolving a preservative in a portion of purified water under continuous stirring to obtain a clear solution, followed by the sequential addition of buffering agents, a surfactant, and a viscosity-modifying polymer with continued stirring until clarity is achieved. An anti-foaming agent is then incorporated to form a homogeneous suspension. Thereafter, a glidant and lurasidone hydrochloride are added sequentially under continuous agitation to obtain a uniform, lump-free suspension. Separately, xanthan gum is dispersed in purified water to form a clear, viscous solution, which is subsequently combined with the lurasidone hydrochloride-containing suspension under continuous stirring to yield a homogeneous composition. Sweetening agent and flavoring agents are then added with further addition of water to obtain the final suspension. The order of addition is critical, and particularly, incorporation of lurasidone hydrochloride prior to the addition of xanthan gum is essential to ensure uniform drug distribution and to prevent premature viscosity build-up that can impede API dispersion.

A process for preparing an oral pharmaceutical suspension comprising lurasidone hydrochloride, the process comprising:
(a) dissolving a preservative in purified water under stirring to form a clear solution;
(b) sequentially adding one or more buffering agents, a surfactant, and a polymeric viscosity-modifying agent to the solution of step (a) under stirring to obtain a clear mixture;
(c) incorporating an antifoaming agent into the mixture of step (b) to form a homogeneous suspension;
(d) sequentially adding a glidant and lurasidone hydrochloride to the suspension of step (c) under continuous stirring to obtain a lump-free, homogeneous suspension;
(e) separately dispersing xanthan gum in purified water under stirring to form a viscous solution;
(f) combining the xanthan gum solution of step (e) with the suspension obtained in step (d) under continuous stirring to form a homogeneous suspension; and
(g) optionally adding one or more sweetening agent and flavoring agents, with further addition of water, to obtain the oral pharmaceutical suspension,
wherein lurasidone hydrochloride is added prior to the addition of xanthan gum, and
wherein the order of addition is critical to ensure uniform distribution of lurasidone hydrochloride and reproducible manufacturability of the suspension.

In some embodiments, the present disclosure relates to an oral suspension composition comprising lurasidone or a pharmaceutically acceptable salt thereof in an amount from about 1 mg/mL to about 200 mg/mL and a viscosity from about 1 cps to about 300 cps.

In some embodiments, the present disclosure relates to an oral suspension composition comprising lurasidone or a pharmaceutically acceptable salt thereof in an amount from about 1 mg/mL to about 100 mg/mL and a viscosity from about 1 cps to about 250 cps.

In some embodiments, the present disclosure relates to an oral suspension composition comprising lurasidone or a pharmaceutically acceptable salt thereof in an amount from about 0.1 mg/mL to about 50 mg/mL and a viscosity from about 1 cps to about 200 cps.

In some embodiments, the present disclosure relates to an oral suspension composition comprising lurasidone or a pharmaceutically acceptable salt thereof in an amount from about 0.1 mg/mL to about 20 mg/mL and a viscosity from about 1 cps to about 250 cps.

In some embodiments, the present disclosure relates to an oral suspension composition comprising about 0.1 mg/mL to about 10 mg/mL lurasidone or a pharmaceutically acceptable salt thereof and one or more pharmaceutically acceptable excipients, wherein the pH of the composition is from about 2 to about 6 and the viscosity from about 1 cps to about 200 cps.

In some embodiments, the dose of lurasidone is administered to a patient in need thereof in an oral suspension described herein is in the range of about 10 mg to about 150 mg per day. In some embodiments, the dose of lurasidone administered to a patient in need thereof in a oral suspension described herein is in the range of from about 1 mL to about 15 mL per day.

In some embodiments, the present invention relates to a stable oral liquid composition comprising lurasidone fumarate, about 0.5% w/v to about 3% w/v of solubilizing agent, about 0.1% w/v to about 2% w/v of salt forming agent, about 0.% w/v to about 3% w/v of one or more suspending agent, about 0.1% w/v to about 5% w/v of wetting agent, about 0.1% w/v to about 5% w/v of anti-foaming agent, about 0.1% w/v to about 5% w/v of pH modifier, about 0.05% to about 1% w/v sweetening agent, about 0.01% w/v to about 1% w/v of flavoring agent, about 0.1% w/v to about 5% w/v of preservative, about 0.1% w/v to about 5% w/v of buffering agent and vehicle; wherein the pH of the liquid composition ranges from about 2.0 to about 6.0.

In another embodiment, the present invention relates to a stable oral liquid composition comprising:
(a) about 0.1 to about 10% of lurasidone or its pharmaceutical acceptable salt thereof,
(b) about 0 to about 3% of solubilizing agent,
(c) about 0 to about 2% of salt forming agent,
(d) about 0 to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0 to about 1% of sweetening agent,
(j) about 0 to about 1% of flavoring agent and
(k) vehicle and pH of the liquid composition is in the range of 2.0 to 6.0;
wherein the lurasidone or its pharmaceutically acceptable salt is the only active ingredient in the pharmaceutical composition.

In another embodiment, the present invention relates to a stable oral solution comprising:
(a) about 0.1 to about 10% of lurasidone or its pharmaceutical acceptable salt thereof,
(b) about 0 to about 3% of solubilizing agent,
(c) about 0 to about 2% of salt forming agent,
(d) about 0 to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0 to about 1% of sweetening agent,
(j) about 0 to about 1% of flavoring agent and
(k) vehicle and pH of the liquid composition is in the range of 2.0 to 6.0;
wherein the lurasidone or its pharmaceutically acceptable salt is the only active ingredient in the pharmaceutical composition.

In another embodiment, the present invention relates to a stable oral solution comprising:
(a) about 0.1 to about 10% of lurasidone fumarate,
(b) about 0 to about 3% of solubilizing agent,
(c) about 0 to about 2% of salt forming agent,
(d) about 0 to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0-about 1% of sweetening agent,
(j) about 0-about 1% of flavoring agent and
(k) vehicle and pH of the liquid composition is in the range of 2.0 to 6.0; wherein the lurasidone fumarate is the only active ingredient in the pharmaceutical composition.

In another embodiment, the present invention relates to a stable oral suspension comprising:
(a) about 0.1 to about 10% of lurasidone or its pharmaceutical acceptable salt thereof,
(b) about 0 to about 3% of solubilizing agent,
(c) about 0 to about 2% of salt forming agent,
(d) about 0.5% to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0-about 1% of sweetening agent,
(j) about 0-about 1% of flavoring agent and
(k) vehicle and pH of the liquid composition is in the range of 2.0 to 6.0;
wherein the lurasidone or its pharmaceutically acceptable salt is the only active ingredient in the pharmaceutical composition.

In another embodiment, the present invention relates to a stable oral suspension comprising:
(a) about 0.1 to about 10% of lurasidone fumarate,
(b) about 0 to about 3% of solubilizing agent,
(c) about 0 to about 2% of salt forming agent,
(d) about 0.5% to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0 to about 1% of sweetening agent,
(j) about 0 to about 1% of flavoring agent,
(k) vehicle and pH of the liquid composition is in the range of 2.0 to 6.0;
wherein the lurasidone fumarate is the only active ingredient in the pharmaceutical composition.

In another embodiment, the present invention relates to a stable oral suspension comprising:
(a) about 0.1 to about 10% of lurasidone or its pharmaceutical acceptable salt thereof,
(b) about 0.5% to about 3% of solubilizing agent,
(c) about 0.1% to about 2% of salt forming agent,
(d) about 0.5% to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(i) about 0 to about 1% of sweetening agent,
(j) about 0 to about 1% of flavoring agent and
(k) vehicle and pH is in the range of 2.0 to 6.0;
wherein the lurasidone or its pharmaceutically acceptable salt is the only active ingredient in the pharmaceutical composition.

In another embodiment, the present invention relates to a stable oral suspension comprising:
(a) about 0.1 to about 10% of lurasidone or its pharmaceutical acceptable salt thereof,
(b) about 0.5% to about 3% of benzyl alcohol,
(c) about 0.1% to about 2% of fumaric acid,
(d) about 0.5% to about 3% of microcrystalline cellulose, carboxymethylcellulose sodium blend and hydroxyethyl cellulose,
(e) about 0.1% to about 5% of trisodium citrate dihydrate,
(f) about 0.01% to about 1% of sucralose,
(g) about 0.1% to about 1% of flavoring agent,
(h) vehicle and pH is in the range of 2.0-6.0;
wherein the lurasidone or its pharmaceutically acceptable salt is the only active ingredient in the pharmaceutical composition.

In another embodiment, the present invention relates to a stable oral suspension comprising:
(a) about 0.1 to about 10% of lurasidone or its pharmaceutical acceptable salt thereof,
(b) about 0.5% to about 3% of benzyl alcohol,
(c) about 0.1% to about 2% of fumaric acid,
(d) about 0.01% to about 2% of polysorbate 80,
(e) about 0.01% to about 2% of simethicone emulsion 30%,
(d) about 0.5% to about 3% of microcrystalline cellulose, carboxymethylcellulose sodium and hydroxyethyl cellulose,
(e) 0.1-5% of trisodium citrate dihydrate,
(f) 0.01-1% of sucralose,
(g) 0.1-1% of flavor mixed berry
(h) vehicle and pH is in the range of 2-6;
wherein the lurasidone or its pharmaceutically acceptable salt is the only active ingredient in the pharmaceutical composition.

In another embodiment, the present invention relates to a stable oral suspension comprising;
(a) about 0.1-about 10% of lurasidone or its pharmaceutical acceptable salt thereof,
(b) about 0.1% to about 3% of one or more suspending agents,
(c) about 0.01% to about 2% of preservative,
(d) about 0.01% to about 4% of one or more buffering agent,
(e) optionally about 0 to about 2% of wetting agent,
(g) optionally about 0 to about 1% of anti-foaming agent,
(h) optionally about 0-about 1% of sweetening agent,
(f) optionally about 0-about 1% of flavor mixed berry and
(g) vehicle and pH is in the range of 2-6;
wherein the lurasidone or its pharmaceutically acceptable salt is the only active-ingredient in the pharmaceutical composition.

In yet another embodiment, the present invention relates to a stable oral suspension comprising;
(a) about 0.1-about 10% of lurasidone or its pharmaceutical acceptable salt thereof,
(b) about 0.1% to about 3% of xanthan gum,
(c) about 0.01% to about 2% of sodium benzoate,
(d) about 0.01% to about 4% of anhydrous citric acid and trisodium citrate dihydrate,
(e) optionally about 0 to about 2% of polysorbate 80,
(g) optionally about 0-about 1% of sucralose,
(h) optionally about 0-about 1% of flavor mixed berry,
(i) vehicle and pH is in the range of 2-6;
wherein the lurasidone or its pharmaceutically acceptable salt is the only active ingredient in the pharmaceutical composition.

In another embodiment, the present invention relates to a stable oral suspension comprising;
(a) about 0.1 to about 10% of lurasidone or its pharmaceutical acceptable salt thereof,
(b) about 0.5% to about 3% of solubilizing agent,
(c) about 0.1% to about 2% of salt forming agent,
(d) about 0.5% to about 3% of one or more suspending agents,
(e) about 0.1% to about 5% of pH modifier,
(f) optionally about 0 to about 2% of wetting agent,
(g) optionally about 0 to about 1% of anti-foaming agent,
(h) optionally about 0 to about 1% of buffering agent,
(f) optionally about 0 to about 1% of sweetening agent,
(g) optionally about 0 to about 1% of flavor mixed berry and
(h) vehicle and pH is in the range of 2-6;
wherein the molar ratio of lurasidone and salt forming agent is the range of 1:1 to about 1:3 and lurasidone or its pharmaceutical acceptable salt thereof, is only active ingredient in the pharmaceutical composition.

In another embodiment, the present invention relates to a stable oral suspension comprising;
(a) about 0.1 to about 10% of lurasidone or its pharmaceutical acceptable salt thereof,
(b) about 0.5% to about 3% of solubilizing agent,
(c) about 0.1% to about 2% of salt forming agent,
(d) about 0.5% to about 3% of one or more suspending agents,
(e) about 0.1% to about 5% of pH modifier,
(f) about 0 to about 2% of wetting agent,
(g) about 0 to about 1% of anti-foaming agent,
(h) about 0 to about 1% of buffering agent,
(f) about 0 to about 1% of sweetening agent,
(g) about 0-about 1% of flavor mixed berry,
(h) vehicle and pH is in the range of 2-6;
wherein the median diameter D90 or D50 or D10 value is in the range of about 1 μm to about 150 μm when measured by a light scattering particle size analyzer.

In another embodiment, the present invention relates to a stable oral liquid composition comprising about 11.5 mg/mL of lurasidone fumarate, which is equivalent to 9.3 mg/ml of lurasidone base. In another embodiment, the present invention relates to a stable oral liquid composition comprising about 23.0 mg/mL of lurasidone fumarate, which is equivalent to 18.6 mg/ml of lurasidone base. In another embodiment, the present invention relates to a stable oral liquid composition comprising about 46.0 mg/mL of lurasidone fumarate, which is equivalent to 37.2 mg/ml of lurasidone base. In another embodiment, the present invention relates to a stable oral liquid composition comprising about 69.1 mg/mL of lurasidone fumarate, which is equivalent to 55.9 mg/ml of lurasidone base. In another embodiment, the present invention relates to a stable oral liquid composition comprising about 92.0 mg/mL of lurasidone fumarate, which is equivalent to 75.0 mg/ml of lurasidone base. In another embodiment, the present invention relates to a stable oral liquid composition comprising about 138.0 mg/mL of lurasidone fumarate, which is equivalent to 111.7 mg/ml of lurasidone base.

In another embodiment, the present invention relates to a stable oral solution comprising about 11.5 mg/mL of lurasidone fumarate, which is equivalent to 9.3 mg/ml of lurasidone base. In another embodiment, the present invention relates to a stable oral liquid composition comprising about 23.0 mg/mL of lurasidone fumarate, which is equivalent to 18.6 mg/ml of lurasidone base. In another embodiment, the present invention relates to a stable oral liquid composition comprising about 46.0 mg/mL of lurasidone fumarate, which is equivalent to 37.2 mg/ml of lurasidone base. In another embodiment, the present invention relates to a stable oral liquid composition comprising about 69.1 mg/mL of lurasidone fumarate, which is equivalent to 55.9 mg/ml of lurasidone base. In another embodiment, the present invention relates to a stable oral liquid composition comprising about 92.0 mg/mL of lurasidone fumarate, which is equivalent to 75.0 mg/ml of lurasidone base. In another embodiment, the present invention relates to a stable oral liquid composition comprising about 138.0 mg/mL of lurasidone fumarate, which is equivalent to 111.7 mg/ml of lurasidone base.

In another embodiment, the present invention relates to a stable oral suspension comprises about 11.5 mg/mL of lurasidone fumarate, which is equivalent to 9.31 mg/ml of lurasidone base. In another embodiment, the present invention relates to a stable oral liquid composition comprising about 23.0 mg/mL of lurasidone fumarate, which is equivalent to 18.6 mg/ml of lurasidone base. In another embodiment, the present invention relates to a stable oral liquid composition comprising about 46.0 mg/mL of lurasidone fumarate, which is equivalent to 37.2 mg/ml of lurasidone base. In another embodiment, the present invention relates to a stable oral liquid composition comprising about 69.1 mg/mL of lurasidone fumarate, which is equivalent to 55.9 mg/ml of lurasidone base. In another embodiment, the present invention relates to a stable oral liquid composition comprising about 92.0 mg/mL of lurasidone fumarate, which is equivalent to 75.0 mg/ml of lurasidone base. In another embodiment, the present invention relates to a stable oral liquid composition comprising about 138.0 mg/mL of lurasidone fumarate, which is equivalent to 111.7 mg/ml of lurasidone base.

In another embodiment, the present invention relates to an oral liquid pharmaceutical suspension comprising lurasidone or pharmaceutically acceptable salt thereof, wherein the ratio of lurasidone to salt forming agent is in the range of 1:1 to about 1:3.

In another embodiment, the present invention relates to an oral liquid comprising, lurasidone or its pharmaceutically acceptable salts thereof, at a concentration of about 1 mg/mL to about 500 mg/mL; at least one pharmaceutically acceptable liquid vehicle and one or more pharmaceutically acceptable excipients, wherein the composition is in the form of a solution suitable for oral administration and the pH remains within a range of 2.0 to 6.0, when stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 12 months.

In another embodiment, the present invention relates to an oral liquid pharmaceutical composition comprising, lurasidone or its pharmaceutically acceptable salts thereof, at a concentration of about 1 mg/mL to about 500 mg/mL; and at least one pharmaceutically acceptable liquid vehicle, wherein the composition is in the form of a suspension suitable for oral administration and the pH remains within a range of 2.0 to 6.0 when stored at 25° C.±2° C./60%±5% RH or 40° C.±2° C./75%±5% RH or 30° C.±2° C./65%±5% RH or 40° C.±2° C./25%±5% RH for at least 3 months or at 2-8° C. for at least 12 months.

In another embodiment, the present invention relates to oral pharmaceutical suspension; wherein the oral pharmaceutical suspensions are stable for at least 6 months when stored at 25° C.±2° C./60%±5% RH or 40° C.±2° C./75%±5% RH or 30° C.±2° C./65%±5% RH or 40° C.±2° C./25%±5% RH and the level of impurities in the solution are less than 0.5% w/w as measured by HPLC.

In another embodiment, the present invention relates to oral pharmaceutical suspension; wherein the oral pharmaceutical suspensions are stable for at least 6 months when stored at 25° C.±2° C./60%±5% RH or 40° C.±2° C./75%±5% RH or 30° C.±2° C./65%±5% RH or 40° C.±2° C./25%±5% RH and the level of impurities in the solution are less than 1% w/w as measured by HPLC.

In another embodiment, the present invention relates to oral pharmaceutical suspension; wherein the oral pharmaceutical suspensions are stable for at least 6 months when stored at 25° C.±2° C./60%±5% RH or 40° C.±2° C./75%±5% RH or 30° C.±2° C./65%±5% RH or 40° C.±2° C./25%±5% RH and the level of impurities in the solution are less than 2% w/w as measured by HPLC.

In another embodiment, the present invention relates to oral pharmaceutical suspension; wherein the oral pharmaceutical suspensions are stable for at least 6 months when stored at 25° C.±2° C./60%±5% RH or 40° C.±2° C./75%±5% RH or 30° C.±2° C./65%±5% RH or 40° C.±2° C./25%±5% RH and the level of impurities in the solution are less than 3% w/w as measured by HPLC.

In another embodiment, the present invention relates to oral liquid solution; wherein the oral pharmaceutical solutions are stable for at least 6 months when stored at 25° C.±2° C./60%±5% RH or 40° C.±2° C./75%±5% RH or 30° C.±2° C./65%±5% RH or 40° C.±2° C./25%±5% RH and the level of impurities in the solution are less than 0.5% w/w as measured by HPLC.

In some embodiments, the pharmaceutically acceptable salt of lurasidone is lurasidone fumarate and is formed in situ by the reaction of lurasidone free base or its pharmaceutically acceptable salt with a salt forming agent by dissolving both in solubilizing agent to form lurasidone fumarate that is more soluble in aqueous media than lurasidone free base or lurasidone hydrochloride. In some embodiments, lurasidone fumarate and is formed in situ by the reaction of lurasidone free base or its pharmaceutically acceptable salt with a fumaric acid salt forming agent to lurasidone fumarate that is more soluble in aqueous media than lurasidone free base or lurasidone hydrochloride. In some embodiments, the fumarate salt forming agent is fumaric acid. In some embodiments, an excess of the fumaric acid salt forming agent is used to form the fumarate salt in situ. In some embodiments, the amount of fumaric acid used as the salt forming agent is about 1.0 mg/ml to about 10.0 mg/ml. In some embodiments, the amount of fumaric acid used as the salt forming agent is about 1.0 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5.0 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6.0 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7.0 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8.0 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9.0 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, or about 10.0 mg/ml.

In some embodiments, the present invention relates to an oral liquid pharmaceutical composition comprising lurasidone fumarate; wherein lurasidone fumarate is formed by the dissolving the lurasidone base or hydrochloride in solubilizing agent and then treating the solution with a molar excess of fumaric acid. In some embodiments of an oral liquid formulation, the formulation further comprises one or more excipients selected from solubilizing agents, salt forming agents, suspending agent, pH modifiers, buffering agents, wetting agents, surfactants, thickening agents, stabilizers, anti-oxidants, chelating agents, preservatives, flavoring agents, sweetening agents, coloring agents, vehicle and mixtures thereof.

In some embodiments, the pharmaceutically acceptable salt of lurasidone is formed in situ as a concentrate and is subsequently diluted with water to arrive at the final suspension. In some embodiments, the concentrate is formed in about 1% of the final liquid volume, In some embodiments, the concentrate is formed in about 5% of the final liquid volume. In some embodiments, the concentrate is formed in about 7.5% of the final liquid volume. In some embodiments, the concentrate is formed in about 10% of the final liquid volume. In some embodiments, the concentrate is formed in about 12.5% of the final liquid volume. In some embodiments, the concentrate is formed in about 15% of the final liquid volume. In some embodiments, the concentrate is formed in about 20% of the final liquid volume. In some embodiments, the concentrate is formed in about 25% of the final liquid volume. In some embodiments, the concentrate is formed in about 30% of the final liquid volume. In some embodiments, the concentrate is formed in about 35% of the final liquid volume. In some embodiments, the concentrate is formed in about 40% of the final liquid volume. In some embodiments, the concentrate is formed in about 45% of the final liquid volume. In some embodiments, the concentrate is formed in about 50% of the final liquid volume. In some embodiments, the concentrate is formed in about 55% of the final liquid volume. In some embodiments, the concentrate is formed in about 60% of the final liquid volume. In some embodiments, the concentrate is formed in about 65% of the final liquid volume. In some embodiments, the concentrate is formed in about 70% of the final liquid volume. In some embodiments, the concentrate is formed in about 75% of the final liquid volume. In some embodiments, the concentrate is formed in about 80% of the final liquid volume. In some embodiments, the concentrate is formed in about 85% of the final liquid volume. In some embodiments, the concentrate is formed in about 90% of the final liquid volume. In some embodiments, the concentrate is formed in about 95% of the final liquid volume.

In some embodiments, the ultrasonic agitation is carried out with any suitable sonication device such as those described in U.S. Pat. Nos. 5,471,001; 6,960,256, or an ultrasonic cleaning tank such as described in U.S. Pat. No. 3,516,645. Such devices are well known in the industry. The use of ultrasonic energy in conjunction with a solvent for cleaning workpieces is well established in the art. Cleaning apparatus of this type have been described, for instance, in U.S. Pat. Nos. 2,845,077; 3,293,456; 3,318,578; and 3,651,352.

By “sonication” it is meant that electrical energy is converted to physical vibrations (sound energy) which are applied to solutions, suspensions, or particles. In some embodiments, the sonication device has a sonication horn or probe that is inserted into the system of interest to emit sonic energy into the solution. In some embodiments, the sonicating device is operated at a frequency between about 1 kHz and about 10 MHz or between about 1 kHz and about 100 kHz or between about 20 kHz and about 40 kHz or any range or combination of ranges therein. In some embodiments, the frequency is modulated slightly around a target frequency. In some embodiments, the sonicating device is operated at a frequency of about 40 kHz with a modulation of about ±1 kHz. In some embodiments, other mixing devices such as homogenizers, blenders, or other stirring devices are used while subjecting the liquid to ultrasonic agitation.

Described herein is a process for preparing oral lurasidone fumarate suspension, the process comprising:
(i) dissolving lurasidone or its pharmaceutically acceptable salt in non-aqueous media to form a first mixture;
(ii) dissolving a salt forming agent to aqueous solvent to form a second mixture;
(iii) mixing first mixture and second mixture results in a suspension by forming in-situ salt of lurasidone.
(iv) subjecting the above suspension to ultrasonic agitation at a frequency of between about 10 kHz and about 100 kHz or physical agitation or physical mixing to reduce the size of particles of suspension.
   wherein the molar ratio of lurasidone and salt forming agent is the range of 1:1 to about 1:3.

Described herein is a process for preparing oral lurasidone fumarate suspension, the process comprising:
(i) dissolving a salting agent to aqueous solvent to form a first mixture;
(ii) dissolving lurasidone or its pharmaceutically acceptable salt in non-aqueous media to form a second mixture;
(iii) mixing first mixture and second mixture results in a suspension by forming in-situ salt of lurasidone.
(iv) subjecting the above suspension to ultrasonic agitation at a frequency of between about 10 kHz and about 100 kHz or physical agitation or physical mixing to reduce the size of particles of suspension.

In some embodiments, the present invention is oral suspension comprising lurasidone fumarate having a median particle diameter D90 or D50 or D10 value of between 1 μm and 150 μm as measured by a particle size analyzer; wherein the suspension is made by mixing Lurasidone or its pharmaceutical acceptable salts thereof dissolved in benzyl alcohol and aqueous Fumaric acid between 1 minute and 1 hour, thereby forming the suspension comprising particles comprising lurasidone fumarate.

In some embodiments, the present invention is oral suspension comprising Lurasidone fumarate having a median diameter D90 or D50 or D10 value is in the range of about 1 μm to about 150 μm as measured by a light scattering particle size analyzer, and the oral suspension is made by a process comprising:
(i) dissolving lurasidone or its pharmaceutical acceptable salts thereof in benzyl alcohol;
(ii) adding aqueous Fumaric acid to the above solution to form a first mixture; and
(iii) subjecting the first mixture to ultrasonic agitation at a frequency of between about 10 kHz and about 100 kHz or physical agitation or physical mixing, thereby forming a second mixture comprising lurasidone fumarate.

In yet another embodiment, the present invention relates to an oral pharmaceutical suspension comprising lurasidone or its pharmaceutically acceptable salts thereof; wherein the particle size range of the lurasidone or its pharmaceutically acceptable salts thereof is in the range of 0.1-200 microns.

In some embodiments, the suspension disclosed herein comprises lurasidone fumarate particles having a D90 value between about 20 μm and about 180 μm. In some embodiments, the suspension disclosed herein comprises lurasidone fumarate particles having a D90 value between about 20 μm and about 150 μm. In some embodiments, the suspension disclosed herein comprises lurasidone fumarate particles having a D90 value between about 25 μm and about 100 μm. In some embodiments, the suspension disclosed herein comprises lurasidone fumarate particles having a D90 value of about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, or about 60 μm.

In some embodiments, the present invention relates to process for preparing a stable oral suspension composition comprising following steps;
(i) Heating the solubilizing agent to a temperature between 50-80° C.;
(ii) Dissolving lurasidone or its pharmaceutically acceptable salt in preheated solubilizing agent of above step (i);
(iii) Salt forming agent is added to 50% preheated water (50-80° C.) and mixed until clear solution is formed;
(iv) Solution of step (ii) transferred to step (iii) and stirred for 20 minutes. The resulting solution cooled to 20-25° C. and homogenized for 45 minutes;
(v) Sweeting agent and flavoring agent are added to solution of Step (iv);
(vi) One or more suspending agents are added to purified water with continuous stirring forming a homogenized suspension;
(vii) The homogenized suspension of step (vi) is transferred to clear solution of step (v) and remaining purified water is added with continuous stirring resulting in a homogenized suspension;
(viii) pH modifier added to above homogenized suspension of step (vii) to have of pH of 2-6 in the homogenized suspension.

In some embodiments, the present invention relates to process for preparing a lurasidone oral suspension, the process comprising:
(i) preparing an aqueous mixture comprising lurasidone hydrochloride;
(ii) preparing a solution comprising a suspending agent;
(iii) combining the solution of step (ii) with the aqueous mixture of step (i) to form a homogeneous suspension; and
(iv) adjusting the pH of the homogeneous suspension obtained in step (iii) to a pH ranging from about 2.0 to about 3.0.

In some embodiments, the pharmaceutical composition of present application is filled into suitable pharmaceutically acceptable container, wherein the pharmaceutically acceptable container is selected from the group consisting of bottle, infusion bag, vial, prefilled syringe, syringe and ampoule.

In some embodiments, the pharmaceutically acceptable container is a bottle, wherein the bottle is selected from group consisting of glass bottle or plastic bottle, wherein glass bottle is selected from group consisting of Type I, II and III borosilicate glass bottles, wherein the glass bottle may be amber color glass bottle or clear glass bottle.

In another embodiment, the pharmaceutically acceptable container is a bottle, wherein the bottle is selected from the group consisting of high-density polyethylene (HDPE) bottle, polyethylene terephthalate (PET) and polypropylene (PP), wherein the plastic bottle may be amber color, white opaque or translucent plastic bottle.

The glass and HDPE bottles are available in 10 mL, 2010 mL, 3010 mL, 6010 mL, 10010 mL, 12010 mL, 15010 mL, 25010 mL & 500 mL fill volumes, among others.

In some embodiments, the pharmaceutical composition of present application are packed in a kit comprising a bottle with child resistant cap, adapter and dosing syringe.

### Stability

As used herein, the term “stable” is defined as no more than about 10% loss of lurasidone under typical commercial storage conditions. In certain embodiments of the present invention, the degradation related loss of lurasidone is no more than about 5%, no more than about 4%, no more than about 3% loss, no more than about 2% loss, no more than about 1% loss, under typical commercial storage conditions.

In some embodiments, the oral liquid compositions of the invention retains at least about 90% of the potency of lurasidone after storing the composition at 25° C.±2° C./60%±5% RH or 40° C.±2° C./75%±5% RH or 30° C.±2° C./65%±5% RH or 40° C.±2° C./25%±5% RH condition for at least 6 months. In certain embodiments, the solution retains at least about 95% of the potency of lurasidone after storing the solution at 2-8° C. for at least twelve months.

In some embodiments, the present invention provides stable oral solution which are stable for at least 3 months or at least 6 months, or at least 9 months, or at least 12 months, at 25° C.±2° C./60%±5% RH or 40° C.±2° C./75%±5% RH or 30° C.±2° C./65%±5% RH or 40° C.±2° C./25%±5% RH storage conditions.

In some embodiments, the present invention provides stable oral solutions which are stable for at least 6 months, or at least 9 months or at least 12 months or at least 18 months or at least 24 months, at 2-8° C. storage conditions.

In some embodiments, the present invention provides stable oral liquid compositions, wherein the level of total impurities in the composition is less than about 5% w/w, preferably less than about 3% w/w, more preferably less than about 1% w/w, more preferably less than about 0.5% w/w as measured by HPLC.

In another embodiment, the level of any unknown impurity in the inventive oral liquid pharmaceutical compositions resulting from the degradation of lurasidone is less than about 5% (w/w), preferably less than about 3% (w/w), preferably less than about 1% (w/w), preferably less than about 0.5% (w/w), preferably less than about 0.15% (w/w) and more preferably less than about 0.1% (w/w) as measured by HPLC.

### Dosage and Administration

In one embodiment, the dose of lurasidone is in the range of from about 0.1 mg/kg/day to about 100 mg/kg/day, preferably in the range from about 0.5 mg/kg/day to about 80 mg/kg/day and more preferably in the range from about 1.0 mg/kg/day to about 60 mg/kg/day.

The dosage levels can be dependent on the nature of the condition, drug efficacy, the condition of the patient, the judgment of the practitioner, and the frequency and mode of administration. The unit dosage forms can be administered to achieve any daily amount described herein, such as by administering one to five times daily (e.g., one, two, three, four, or five times daily).

In some embodiments, each administration of the pharmaceutical composition provides a dosage amount of 5 mg, 10 mg, 15 mg, 20 mg, 25 mg, 30 mg, 35 mg, 40 mg, 45 mg, 50 mg, 55 mg, 60 m g, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg.

In some aspects, an effective amount of the pharmaceutical composition may be administered for treatment of CNS disorders in a human, including schizophrenia and depressive episodes associated with bipolar disorder.

As used herein, “to treat” a condition or “treatment” of the condition is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### EXAMPLES

The following examples are exemplary and not intended to be limiting. The above disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

### Abbreviations

| | |
|---|---|
| API | active pharmaceutical ingredient |
| HPLC | high-performance liquid chromatography |
| h | hour/hours |
| min | minute/minutes |
| QS | quantity sufficient |
| RSD | Relative standard deviation |
| RH | relative humidity |
| USP | United States Pharmacopoeia |

### Example 1: In-Situ Salt Formation

Based on pKa, different salt forming agents were evaluated. The following table summarizes the list of trials and their observations, where the examples were prepared by the manufacturing procedure below.

TABLE 1
In-situ salt formation trials

| S. No. | Active ingredient | Salting agent | Remarks |
|---|---|---|---|
| 1 | Lurasidone | Tartaric acid | No salt formed |
| 2 | | Succinic acid | No salt formed |
| 3 | | Acetic acid | No salt formed |
| 4 | | Ortho-phosphoric acid | No salt formed |
| 5 | | Maleic acid | No salt formed |
| 6 | | Malic acid | No salt formed |
| 7 | | Sodium pamoate | No salt formed |
| 8 | | Fumaric acid | Salt formed |

### Manufacturing Procedure for In-Situ Salt Formation:

(i) The benzyl alcohol was heated to a temperature between 50-80° C.;
(ii) The lurasidone or pharmaceutically acceptable salt thereof was dissolved with preheated benzyl alcohol from above step (i);
(iii) Salting agent (Fumaric acid) was added to 50% preheated water (50-80° C.) and mixed until a clear solution was formed.

### Example 1A: Analytical Method: Particle Size Distribution (PSD)

A Malvern Mastersizer 3000 (laser diffraction) integrated with a Hydro MV unit using water as the dispersant was configured. The suspension bottle was gently agitated for 30 seconds, and the sample was gradually introduced into the Hydro MV module containing the dispersant until the obscuration was brought within the specified range (nominal range: 8-10%). The measurement was initiated by pressing the start button, and particle size distribution values at D10, D50 (median), and D90 were recorded.

### Example 1B: HPLC Analysis for % Lurasidone Assays

High-performance liquid chromatography (HPLC) equipped with quaternary gradient pumps, UV detector with data recorder, and integrator software was employed. 10 μL samples were injected into a X-bridge C18 column (150 mm length×4.6 mm ID×3.5 μm particle size), using isocratic program with 0.2% ammonia solution pH 8.5 buffer and acetonitrile in the ratio of 20:80 (v/v) in pump A (mobile phase).

The chromatographic parameters were fixed at a flow rate of 1.2 mL, a column thermostat was used at 25° C., and the UV response was monitored at 232 nm.

A 48 ppm concentration standard and 45 ppm concentration samples of Lurasidone injected to calculate the % assays of Lurasidone. Retention times of Lurasidone were observed at about 6 to 7 min. The method was found to be free from blank and placebo interference, specific to Lurasidone, precise, accurate, and robust.

### HPLC Analysis for Related Substance Assays

High-performance liquid chromatography (HPLC) equipped with quaternary gradient pumps, UV detector with data recorder, and integrator software was employed. 25 μL samples were injected into a X-bridge C18 column (150 mm length×4.6 mm ID×3.5μ particle size), using a gradient program with 0.2% ammonia solution pH 8.5 buffer in pump A (mobile phase A) and acetonitrile in pump B (mobile phase B). The gradient program was set as follows:

| Time (min) | Mobile Phase A %: Mobile Phase B % (v/v) |
|---|---|
| 0 | 55:45 |
| 6 | 55:45 |
| 30 | 28:72 |
| 38 | 10:90 |
| 43 | 10:90 |
| 45 | 55:45 |
| 50 | 55:45 |

The chromatographic parameters were fixed at a flow rate of 1.2 mL, a column thermostat was used at 35° C., and the UV response was monitored at 232 nm.

A diluted standard (0.96 ppm concentration) and samples (372 ppm concentration) were injected. A placebo solution was injected to disregard the placebo peaks during quantification of impurities.

Relative retention times of the following impurities: piperazin benzthiazole impurity, piperazinium impurity, diastereomer-2 impurity and imine impurity were observed as 0.07, 0.12, 0.94 and 0.97, respectively, considering lurasidone as the main analyte which elutes at about 29 min.

### Example 2: Lurasidone Oral Liquid Compositions

TABLE 2

| S. | | | Composition 1 | | Composition 2 | |
|---|---|---|---|---|---|---|
| No. | Ingredients | Category | mg/mL | % w/v | mg/mL | % w/v |
| 1 | Lurasidone | API | 10.00 | 1.0 | 10.00 | 1.0 |
| 2 | Benzyl alcohol | Solubilizing | 18.00 | 1.8 | 18.00 | 1.8 |
| | | agent | | | | |
| 3 | Fumaric acid | Salt forming | 4.72 | 0.472 | 4.72 | 0.472 |
| | | agent | | | | |
| 4 | Polysorbate 80 | Wetting agent | — | — | 1.0 | 0.1 |
| 5 | Simethicone | Anti-foaming | — | — | 0.1 | 0.01 |
| | Emulsion 30% | agent | | | | |
| 6 | Microcrystalline | Suspending | 10.0 | 1.0 | 10.0 | 1.0 |
| | Cellulose and Sodium | agent | | | | |
| | Carboxymethyl | | | | | |
| | cellulose | | | | | |
| | (Avicel ® RC 591) | | | | | |
| 7 | Hydroxyethyl | Suspending | 1.5 | 0.15 | 1.5 | 0.15 |
| | cellulose | agent | | | | |
| 8 | Sucralose | Sweetening | 1.000 | 0.100 | 1.000 | 0.100 |
| | | agent | | | | |
| 9 | Sodium citrate | pH modifier | — | — | 3.8 | 0.38 |
| | dihydrate | | | | | |
| 10 | Mixed berry flavor | Flavoring agent | 4.000 | 0.400 | 4.000 | 0.400 |
| 11 | Water | Vehicle | Q.S to | — | Q.S to | — |
| | | | 1 mL | | 1 mL | |

| | | | |
|---|---|---|---|
| 12 | pH of final suspension | 2.6 | 4.0 |

### Manufacturing Procedure of Composition 1:

(i) Benzyl alcohol was to a temperature between 50-80° C.;
(ii) Lurasidone or its pharmaceutically acceptable salt was dissolved with preheated benzyl alcohol of above step (i);
(iii) Fumaric acid was added to 50% preheated water (50-80° C.) and mixed till clear solution is formed.
(iv) Clear solution of step (ii) transferred to step (iii) and stirred for 20 minutes to form a suspension. The resulting suspension cooled to 20-25° C. and homogenized for 45 minutes.
(v) Sweetening and flavoring agents were added to solution of step (iv).
(vi) A blend of microcrystalline cellulose and carboxymethylcellulose sodium (Avicel® RC 591) and hydroxyethyl cellulose were added to 40% of total purified water with continuous stirring for suitable time to form a homogenized suspension.
(vii) The homogenized suspension of step (vi) is transferred to step (v) and remaining purified water was added and stirred continuously until a homogenized suspension was formed.

### Manufacturing Procedure of Composition 2:

(i) Benzyl alcohol was heated to a temperature between 50-80° C. in a vessel;
(ii) Lurasidone or its pharmaceutically acceptable salt thereof was dissolved in pre-heated benzyl alcohol of above step (i) to form a clear solution;
(iii) Fumaric acid was added to 50% preheated water (50-80° C.) and mixed until clear solution was formed.
(iv) Clear solutions of step (ii) and step (iii) were mixed and stirred for 20 minutes to form suspension which is then cooled to 20-%25° C. and homogenized for 45 minutes.
(v) Polysorbate 80 and simethicone emulsion were added to step (iv) and mixed for suitable time. The suspension was homogenized for 15 minutes.
(vi) Hydroxyethyl cellulose was added to step (v) and mixed for 45 minutes.
(vii) A blend of microcrystalline cellulose and carboxymethylcellulose sodium (Avicel® RC 591) was mixed in purified water with continuous stirring and then added to above step
(vi) to homogenize for suitable time.
(viii) Trisodium citrate was added to above step (vii), followed by addition of sucralose and berry flavor and stirred for suitable time.
(ix) The suspension of above step (viii) was made up to required volume and the suspension was homogenized for suitable time.

### Example 3: Composition of Lurasidone Base Oral Suspension is Set Forth in Table 3

TABLE 3

| S. | Composition 3 | | | |
|---|---|---|---|---|
| No. | Ingredients | Category | mg/mL | % w/v |
| 1 | Lurasidone | API | 10.00 | 1.000 |
| 2 | Xanthan gum | Suspending agent | 3.00 | 0.300 |
| 3 | Sodium benzoate | Preservative | 1.00 | 0.100 |
| 4 | Anhydrous citric acid | Buffering agent | 0.457 | 0.046 |
| 5 | Trisodium citrate | Buffering agent | 5.180 | 0.518 |
| | dihydrate | | | |
| 6 | Polysorbate 80 | Wetting agent | 1.000 | 0.100 |
| 7 | Sucralose | Sweetening agent | 1.000 | 0.100 |
| 8 | Flavor mixed berry | Flavoring agent | 4.000 | 0.400 |
| 9 | Water | Vehicle | Q.S to 1 mL | — |

pH of final suspension: 3.84

### Manufacturing Procedure of Composition 3:

(i) Sodium benzoate was dissolved in purified water (50% of total volume) under stirring, to form a clear solution.
(ii) Anhydrous citric acid and trisodium citrate, followed by polysorbate 80 were added to above step (i) with continuous stirring, to form a clear solution.
(iii) Lurasidone was added to above step (ii) and stirred until lump free suspension observed.
(iv) Homogenization of above suspension was performed for 45 minutes.
(v) Xanthan gum was dissolved in purified water (30% of total volume) under stirring, to form a clear solution.
(vi) The solution of above step (v) was mixed with suspension of step (iv) and stirred until uniform suspension was formed.
(vii) Sucralose and berry flavor were added to above step (vi) suspension and stirred for suitable time stirred for suitable time.
(viii) The suspension of above step (vii) was made up to required volume and the suspension was homogenized for suitable time till uniform suspension was formed.

### Example 4: Stability Data of Composition 1

TABLE 4

| | | 40° C./75% RH | | | 25° C./60% RH | |
|---|---|---|---|---|---|---|
| Test | Initial | 1 Month | 2 Months | 3 Months | 1 Month | 3 Months |
| Assay | 101.9 | 101.7 | 99.6 | 100.3 | 101.6 | 98.5 |
| pH | 2.66 | 2.66 | 2.69 | 2.70 | 2.66 | 2.71 |

% Drug Release at different time points (in minutes)

| | | | | | | |
|---|---|---|---|---|---|---|
| 5 minutes | 85 | 81 | 98 | 92 | 84 | 85 |
| 10 minutes | 90 | 87 | 100 | 91 | 88 | 92 |
| 15 minutes | 91 | 89 | 100 | 92 | 88 | 94 |
| 30 minutes | 93 | 89 | 100 | 95 | 89 | 96 |
| 45 minutes | 92 | 90 | 100 | 95 | 89 | 96 |

Related Substances (% w/w)

| | | | | | | |
|---|---|---|---|---|---|---|
| Piperazin | ND | ND | ND | ND | ND | ND |
| Benzthiazole | | | | | | |
| Piperazinium | ND | ND | ND | ND | ND | ND |
| Impurity | | | | | | |
| Diastereomer- | 0.03 | 0.03 | 0.03 | 0.03 | 0.03 | 0.03 |
| 2 | | | | | | |
| Imine | ND | ND | ND | ND | ND | ND |
| Impurity | | | | | | |
| Highest | 0.02 | 0.02 | 0.02 | 0.02 | 0.02 | 0.01 |
| unknown | | | | | | |
| Total | 0.07 | 0.07 | 0.05 | 0.05 | 0.07 | 0.04 |

The composition was physically and chemically stable for at least 3 months when stored at 25° C./60% RH & 40° C./75% RHK with no significant change in assay.

### Example 5: Comparison of Dissolution Profiles of LATUDA®, Composition 1 & 3

TABLE 5

| | | Lurasidone fumarate | Lurasidone base |
|---|---|---|---|
| | | suspension | suspension |
| Time | LATUDA ® 40 | 10 mg/mL | 10 mg/mL |
| (minutes) | mg | Composition 1 | Composition 3 |
| 5 | 34 | 85 | 8 |
| 10 | 75 | 90 | 12 |
| 15 | 84 | 91 | 14 |
| 30 | 88 | 93 | 19 |
| 45 | 92 | 92 | 23 |
| 60 | 94 | 94 | 25 |

### Dissolution Profiles of LATUDA®, Composition 1 and 3:

When tested by using USP apparatus II (paddle); volume 900 mL of dissolution media McIlvaine buffer, pH 3.8 for 60 minutes at 37±0.5° C. and stirred at 50 RPM, the dissolution profiles of LATUDA®, Composition 1 and 3 are provided in the above Table 5. Samples of 10 mL were withdrawn at 5, 10, 15, 30, 45, and 60 minutes from dissolution media. 10 mL dissolution media was added after each sample withdrawal. Withdrawn samples were filtered and analyzed using HPLC system with UV spectrophotometer at a wavelength 232 nm.

TABLE 6
Dissolution profile of Composition 2 vs Reference
composition in pH 4.5 acetate buffer:

| | | |
|---|---|---|
| | | Composition 2 |
| | Reference composition | 10 mg/mL |
| Time Point | LATUDA ® | (Initial-pH 4.0) |

| (minutes) | Average | % RSD | Average | % RSD |
|---|---|---|---|---|
| 5 | 28 | 4 | 74 | 1 |
| 10 | 45 | 3 | 85 | 1 |
| 15 | 51 | 1 | 91 | 2 |
| 30 | 53 | 1 | 93 | 1 |
| 45 | 54 | 1 | 94 | 1 |
| 60 | 55 | 1 | 94 | 0 |

### Dissolution Profiles of LATUDA® and Composition 2:

When tested by using USP apparatus II (paddle); volume 1000 mL of dissolution media (pH 4.5 acetate buffer) for 60 minutes at 37±0.5° C. and stirred at 75 RPM, the dissolution profiles of LATUDA® and Composition 2 are provided in the above Table 6. Samples of 10 mL were withdrawn at 5, 10, 15, 30, 45, and 60 minutes from dissolution media. 10 mL dissolution media was added after each sample withdrawal. Withdrawn samples were filtered and analyzed using HPLC system with UV spectrophotometer at a wavelength 232 nm.

### Example 6: Composition of Lurasidone HCl Oral Suspension

TABLE 7

| | Composition 4 | | | |
|---|---|---|---|---|
| S.No. | Ingredients | Category | mg/mL | % w/v |
| 1. | Lurasidone HCl | API | 10.00* | 1.0 |
| 2. | Benzyl alcohol | Preservative | 5.00 | 0.50 |
| 3. | Citric acid | Buffering Agent | 13.00 | 1.30 |
| 4. | Disodium hydrogen | Buffering agent | 0.50 | 0.05 |
| | phosphate | | | |
| 5. | Poloxamer (P188) | Wetting agent | 1.00 | 0.10 |
| 6. | HPMC | Suspending agent | 10.00 | 1.00 |
| | (Methocel ® E15) | | | |
| 7. | Simethicone 30% | Antifoaming agent | 2.00 | 0.20 |
| | emulsion | | | |
| 8. | Colloidal silicone | Anti-caking agent | 2.00 | 0.20 |
| | dioxide | | | |
| 9. | Xanthan gum | Suspending agent | 3.00 | 0.30 |
| 10. | Sucralose | Sweetening agent | 3.00 | 0.30 |
| 11. | Flavor mixed berry | Flavoring agent | 10.00 | 1.00 |
| 12. | Masking 2521 | Bitter blocker | 8.50 | 0.85 |
| 13. | Purified water | Vehicle | Q.S to 1 mL | — |

Target pH of final suspension: 2.4
*10 mg of Lurasidone hydrochloride is equivalent to 9.31mg of Lurasidone base.

Manufacturing process:
(i) Benzyl alcohol was added to 50% of purified water in a vessel and stirred continuously until clear solution was obtained.
(ii) Citric acid monohydrate, disodium hydrogen phosphate dihydrate, poloxamer, hypromellose were added sequentially to above step (i) and stirred continuously, until clear solution was obtained.
(iii) Simethicone 30% emulsion was added to above step (ii) and stirred continuously, until a homogenous suspension was observed.
(iv) Colloidal silicon dioxide and Lurasidone HCl were added sequentially to above step (iii) and stirred continuously, until a lump free homogenous suspension was formed.
(v) Xanthan gum was added to about 30% of purified water from total quantity in separate vessel and stirred continuously until clear lump free viscous solution was obtained.
(vi) Xanthan gum solution of above step was transferred to step iv and stirred continuously, until a homogenous suspension was observed.
(vii) Sucralose, masking 2521 and mixed berry flavor were added sequentially to above step (vi) and stirred continuously with addition of water, until a homogenous suspension was observed.
(viii) The pH of the final suspension was found to be 2.4.

### Dissolution Profile of Composition 4:

Dissolution testing was conducted for Composition 4 at initial stage, and for samples after storage at 40° C./75% RH and 60° C. for one month by using USP apparatus II (paddle); volume 900 mL of dissolution media (pH 3.8 McIlvaine buffer) for 45 minutes at 37±0.5° C. and stirring at 50 RPM. The dissolution profile of Compositions 4 are provided in the above Table 8. Samples of 10 mL were withdrawn at 5, 10, 15, 30, and 45 minutes from dissolution media. 10 mL dissolution media was added after each sample withdrawal. Withdrawn samples were filtered and analyzed using HPLC system with UV spectrophotometer at a wavelength 232 nm.

TABLE 8

| Time Points | Composition 4 | | |
|---|---|---|---|
| (minutes) | Initial | 40° C./75% RH (1 Month) | 60° C. (1 Month) |
| 5 | 98 | 100 | 100 |
| 10 | 97 | 100 | 101 |
| 15 | 97 | 100 | 101 |
| 30 | 98 | 101 | 102 |
| 45 | 98 | 101 | 101 |

### Example 7: Lurasidone HCl Suspension Compositions

TABLE 9

| | | | | |
|---|---|---|---|---|
| | | | Composition 5 | Composition 6 |
| S.No. | Ingredients | Category | mg/mL | mg/mL |

Phase-1

| | | | | |
|---|---|---|---|---|
| 1. | Lurasidone | API | 10.00 | 10.00 |
| | hydrochloride | | | |
| 2. | Benzyl alcohol | Preservative | 5.00 | 5.00 |
| 3. | Citric acid | Buffering | 13.00 | 13.00 |
| | monohydrate | agent | | |
| 4 | Disodium | Buffering | 0.52 | 3.74 |
| | hydrogen | agent | | |
| | phosphate | | | |
| | dihydrate | | | |
| 5 | Poloxamer | Wetting agent | 5.00 | 5.00 |
| | P188 | | | |
| 6. | Simethicone | Antifoaming | 2.00 | 2.00 |
| | emulsion 30% | agent | | |
| 7. | Hypromellose | Suspending | 10.00 | 10.00 |
| | E15 | agent | | |

Phase-2

| | | | | |
|---|---|---|---|---|
| 8. | Xanthan Gum | Suspending | 3.00 | 3.00 |
| | | agent | | |
| 9. | Sucralose | Sweetening | 3.00 | 3.00 |
| | | agent | | |
| 10. | Propylene | Co-solvent | NA | 50.00 |
| | glycol | | | |
| 11. | Polyethylene | Co-solvent | NA | 60.00 |
| | glycol 400 | | | |
| 12. | Flavor mixed | Flavoring | 10.00 | 10.00 |
| | berry | agent | | |
| 13. | Masking 2521 | Bitter blocker | 8.50 | 8.50 |
| 14. | Purified water | Vehicle | q.s to 1 mL | q.s to 1 mL |

| | | |
|---|---|---|
| PH | 2.4 | 3.0 |

### Manufacturing Process of Compositions 5 and 6:

### Phase-1:

1. Benzyl alcohol was dissolved in purified water (~50% of total volume) and stirred continuously to form a clear solution.
2. Citric acid monohydrate, disodium hydrogen phosphate dihydrate and poloxamer P188 were added sequentially to above step 1 and stirred continuously to form a clear solution.
3. Simethicone emulsion 30% was added to above step 2 and stirred continuously to form uniform suspension.
4. Lurasidone HCl and Hypromellose E15 were added sequentially to above step 3 and stirred continuously to form uniform suspension.
5. The above suspension was homogenized for 10 minutes to form uniform suspension.

### Phase-2

6. Xanthan gum was dissolved in purified water (~30% of total volume) and stirred continuously to form a clear solution.
7. The clear solution of step 6 was transferred to step 5 and stirred continuously to form uniform suspension.
8. Sucralose was added to above step 7 and stirred continuously to form an uniform suspension.
9. Flavor mixed berry and masking 2521 were added to above step 8 and stirred continuously to form uniform suspension.
10. Propylene glycol and polyethylene glycol were added in above suspension of step 8, where applicable, to form a uniform suspension.
11. The volume of above suspension was made up to required volume with water and stirred to form uniform suspension.

### Example 8: Lurasidone HCl Suspension Composition

TABLE 10

| | Composition 7 | | |
|---|---|---|---|
| S.No. | Ingredients | Category | mg/mL |

Phase-1

| | | | |
|---|---|---|---|
| 1. | Lurasidone Hydrochloride | API | 10.00 |
| 2. | Sodium benzoate | Preservative | 1.00 |
| 3. | Anhydrous citric acid | Buffering agent | 0.457 |
| 4. | Trisodium citrate | Buffering agent | 5.18 |
| 5. | Polysorbate 80 | Wetting agent | 1.00 |

Phase-2

| | | | |
|---|---|---|---|
| 6. | Xanthan gum | Suspending agent | 3.00 |
| 7. | Sucralose | Sweetening agent | 1.00 |
| 8. | Flavor mixed berry | Flavoring agent | 4.00 |
| 9. | Purified water | Vehicle | q.s to 1 mL |

| | |
|---|---|
| pH | 3.8 |

### Manufacturing Process:

### Phase-1:

1. Benzyl alcohol was dissolved in purified water (~50% of total volume) and stirred continuously to form a clear solution.
2. Citric acid anhydrous, trisodium citrate and polysorbate 80 were added sequentially to step 1 and stirred continuously to form a clear solution.
3. The above suspension of step 2 was homogenized for 10 minutes to form uniform suspension.
4. Lurasidone HCl was added to above step 3 and stirred continuously to form a homogenous lump free suspension.

### Phase-2

5. Xanthan gum was dissolved in purified water and stirred continuously to form a clear solution.
6. The clear solution of step 5 was transferred to step 4 and stirred continuously to form uniform suspension.
7. Sucralose, flavor mixed berry and masking 2521 were added to sequentially to step 6 and stirred continuously to form an uniform suspension.

### Example 9: Effect of pH on Dissolution of Lurasidone HCl Oral Suspensions

### Dissolution Profile of Compositions 5, 6 and 7:

Dissolution testing was conducted for Compositions 5, 6 and 7 after storage at 40° C./75% RH for one month by using USP apparatus II (paddle); volume 900 mL of dissolution media (pH 3.8 McIlvaine buffer) for 45 minutes at 37±0.5° C. and stirring at 50 RPM, the dissolution profile of Compositions 5, 6 and 7 are provided in the above Table 11. Samples of 10 mL were withdrawn at 5, 10, 15, 30, and 45 minutes from dissolution media. 10 mL dissolution media was added after each sample withdrawal. Withdrawn samples were filtered and analyzed using HPLC system with UV spectrophotometer at a wavelength 232 nm.

TABLE 11

| Time Point | | | |
|---|---|---|---|
| (minutes) | Composition 5 | Composition 6 | Composition 7 |
| 5 | 100 | 101 | 11 |
| 10 | 100 | 100 | 21 |
| 15 | 100 | 100 | 28 |
| 30 | 101 | 101 | 46 |
| 45 | 101 | 101 | 60 |

Observation: The dissolution of a lurasidone hydrochloride oral suspensions (Compositions 5, 6 and 7) were investigated at different pH levels. The results showed that dissolution performance was maintained within a pH range of about 2.0 to about 3.0. At pH values outside this range, decreased dissolution was observed, which was associated with crystal growth of lurasidone hydrochloride in the suspension.

### Example 10: Lurasidone HCl Suspension Compositions

TABLE 12

| S. | | | Composition 8 | | Composition 9 | |
|---|---|---|---|---|---|---|
| No | Ingredients | Category | mg/mL | % w/v | mg/mL | % w/v |

Phase-I

| | | | | | | |
|---|---|---|---|---|---|---|
| 1. | Lurasidone HCl | API | 10.00 | 1.0 | 10.00 | 1.0 |
| 2. | Sodium benzoate | Preservative | 1.00 | 0.10 | | — |
| 3. | Benzyl alcohol | Preservative | — | — | 5.00 | 0.50 |
| 4. | Citric acid | Buffering agent | 13.00 | 1.30 | 13.00 | 1.30 |
| 5. | Disodium hydrogen | Buffering agent | 0.52 | 0.05 | 0.50 | 0.05 |
| | phosphate | | | | | |
| 6. | Poloxamer (P188) | Wetting agent | 1.00 | 0.10 | 1.00 | 0.10 |
| 7. | Hydroxypropyl | Suspending agent | 10.00 | 1.00 | — | — |
| | methylcellulose | | | | | |
| | (Methocel ® E15) | | | | | |
| 8. | Hydroxyethyl cellulose | Suspending agent | — | — | 2.00 | 0.20 |
| 9. | Simethicone 30% | Antifoaming agent | 2.00 | 0.20 | 2.00 | 0.20 |
| | emulsion | | | | | |
| 10. | Colloidal silicone dioxide | Anticaking agent | 2.00 | 0.20 | — | — |

Phase-II

| | | | | | | |
|---|---|---|---|---|---|---|
| 11. | Xanthan gum | Suspending agent | 2.00 | 0.20 | — | — |
| 12. | Microcrystalline cellulose/ | Suspending agent | — | — | 10.00 | 0.10 |
| | carboxymethylcellulose | | | | | |
| | Sodium (Avicel ® RC 591) | | | | | |
| 13. | Sucralose | Sweetening agent | 3.00 | 0.30 | 3.00 | 0.30 |
| 14. | Flavor mixed berry | Flavoring agent | 10.00 | 1.00 | 10.00 | 1.00 |
| 15. | Masking 2521 | Bitter blocker | 8.50 | 0.85 | 8.50 | 0.85 |
| 16. | Citric acid | Buffering agent | Q.S | Q.S | Q.S | Q.S |
| 17. | Purified water | Vehicle | Q.S to | — | Q.S to | — |
| | | | 1 mL | | 1 mL | |

| | | | |
|---|---|---|---|
| 18. | pH | 2.4 | 2.4 |

### Manufacturing Process of Compositions 8 and 9:

### Phase-I

(i) weighed quantity of preservative (benzyl alcohol/sodium benzoate) was added to 50% of purified water in a vessel and stirred continuously until clear solution was obtained.
(ii) Citric acid monohydrate, disodium hydrogen phosphate dihydrate and poloxamer p188 were added sequentially to above step (i) and stirred continuously, until clear solution was obtained.
(iii) weighed quantity of suspending agent (hydroxypropyl methylcellulose/hydroxyethyl cellulose) was added to above step (ii) and stirred continuously, until clear solution was obtained.
(iv) Simethicone 30% emulsion was added to above step (iii) and stirred continuously, until a homogenous suspension was observed.
(v) Lurasidone HCl were added sequentially to above step (iv) and stirred continuously, until a lump free homogenous suspension was formed.

### Phase-II

(vi) weighed quantity of suspending agent (xanthan gum/microcrystalline cellulose/carboxymethylcellulose sodium) was added to about 30% of purified water from total quantity in separate vessel and stirred continuously until clear lump free viscous solution was obtained.
(vii) the clear solution of above step (vi) was transferred to step (v) and stirred continuously, until a homogenous suspension was observed.
(vii) Sucralose, masking 2521 and mixed berry flavor were added sequentially to above step (vi) and stirred continuously with addition of water, until a homogenous suspension was observed.
(viii) The pH of the final suspension was found to be 2.4.

### Dissolution Profile of Compositions 8 and Composition 9:

Dissolution testing was conducted for Compositions 8 and 9 at initial stage by using USP apparatus II (paddle); volume 900 mL of dissolution media (pH 3.8 McIlvaine buffer) for 45 minutes at 37±0.5° C. and stirring at 50 RPM. The dissolution profile of Compositions 8 and 9 are provided in the above Table 13. Samples of 10 mL were withdrawn at 5, 10, 15, 30, and 45 minutes from dissolution media. 10 mL dissolution media was added after each sample withdrawal. Withdrawn samples were filtered and analyzed using HPLC system with UV spectrophotometer at a wavelength 232 nm.

TABLE 13

| Time Points | | |
|---|---|---|
| (minutes | Composition 8 | Composition 9 |
| 5 | 96 | 97 |
| 10 | 96 | 98 |
| 15 | 97 | 98 |
| 30 | 97 | 98 |
| 45 | 97 | 98 |

Having now fully described this invention, it will be understood by those of ordinary skill in the art that it can be performed within a wide equivalent range of parameters without affecting the scope of the invention or any embodiment thereof. All publications, patent applications and patents disclosed herein are incorporated by reference in their entirety.

What is claimed is:

1. A stable oral liquid pharmaceutical composition comprising:
(a) lurasidone or a pharmaceutically acceptable salt thereof;
(b) at least one pharmaceutically acceptable liquid vehicle; and
(c) optionally, one or more excipients selected from group consisting of suspending agents, preservatives, buffering agents, wetting agents, anti-foaming agents, anti-caking agents, bitter blockers, thickening agents, pH modifiers, anti-oxidants, complexing agents, chelating agents, flavoring agents, sweetening agents, coloring agents or combinations thereof;
wherein the pH of the pharmaceutical composition is in a range from about 1.5 to about 5; and
wherein the pharmaceutical composition is stable for at least 1 month when stored at about 40° C. and about 75% relative humidity (RH).

2. The stable liquid pharmaceutical composition according to claim 1, wherein the composition is a suspension or a solution.

3. The stable liquid pharmaceutical composition according to claim 1, wherein the composition is an aqueous suspension.

4. The stable liquid pharmaceutical composition according to claim 1, wherein the pH of the composition ranges from about 2 to about 3.

5. The stable liquid pharmaceutical composition according to claim 1, wherein the buffering agent is present at a concentration that ranges from about 0.1% w/v to about 5% w/v.

6. The stable liquid pharmaceutical composition according to claim 1, wherein the buffering agent is present, and wherein the buffering agent comprises citric acid monohydrate, anhydrous citric acid, disodium hydrogen phosphate sodium citrate, sodium acetate, sodium phosphate, potassium phosphate, tris, sodium succinate, sodium tartrate, sodium benzoate, gluconic acid, ascorbic acid or a combination thereof.

7. The stable liquid pharmaceutical composition according to claim 1, wherein the suspending agent is present, and wherein a concentration of the suspending agent is in a range of about 0.01% to about 10% w/v.

8. The stable liquid pharmaceutical composition according to claim 1, wherein the suspending agent is present, and wherein the suspending agent comprises xanthan gum, guar gum, acacia gum, gellan gum, carboxymethylcellulose sodium and hydroxy ethylcellulose, microcrystalline cellulose, hydroxypropyl methylcellulose, sodium alginate, methylcellulose, colloidal silica or a combination thereof.

9. The stable liquid pharmaceutical composition according to claim 1, wherein the suspending agent is present, and further wherein the ratio of lurasidone or salt thereof to the suspending agent is in a range of about 1:0.5 to about 1:10.

10. The stable liquid pharmaceutical composition according to claim 1, wherein the preservative is present, and wherein the concentration of the preservative is in a range of 0.1% to about 5% w/v.

11. The stable liquid pharmaceutical composition according to claim 1, wherein the preservative is present and wherein the preservative comprises methyl paraben, propyl paraben, sodium propionate, benzyl alcohol, potassium sorbate, sodium benzoate, butylated hydroxyanisole, butylated hydroxytoluene, chlorobutanol, phenyl ethyl alcohol or a combination thereof.

12. The stable liquid pharmaceutical composition according to claim 1, wherein the wetting agent is present and wherein the concentration of the wetting agent is in a range of 0.01% to about 5% w/v.

13. The stable liquid pharmaceutical composition according to claim 1, wherein the wetting agent comprises poloxamer (P188), polysorbate 80, polyethylene glycols (PEGs), sorbitan monolaurate, polysorbate 20, sodium lauryl sulfate, sorbitan esters, lecithin, or a combination thereof.

14. The stable liquid pharmaceutical composition according to claim 1, wherein the pharmaceutically acceptable liquid vehicle comprises water, ringer's solution, isotonic sodium chloride solution, glycerin, propylene glycol, ethanol, polyethylene glycol or a combination thereof.

15. The stable liquid pharmaceutical composition according to claim 1, wherein the anti-foaming agent is present and wherein the anti-foaming agent comprises simethicone, alcohols, paraffin oils, stearates and glycols or a combination thereof.

16. The stable liquid pharmaceutical composition according to claim 1, wherein the anti-caking agent is present, and wherein the concentration of the anti-caking agent is in a range of 0.01% to about 10% w/v.

17. The stable liquid pharmaceutical composition according to claim 1, wherein the anti-caking agent is present, and wherein the anti-caking agent comprises colloidal silica, magnesium trisilicate, calcium phosphate tribasic, magnesium oxide, magnesium silicate, calcium silicate, talc or a combination thereof.

18. The stable liquid pharmaceutical composition according to claim 1, wherein the sweetening agent is present, and wherein the concentration of the sweetening agent is in a range of 0.01% to about 3% w/v.

19. The stable liquid pharmaceutical composition according to claim 1, wherein the sweetening agent is present, and wherein the sweetening agent comprises sucralose, trehalose, xylose, dextrose, tagatose, glycerol, lactitol, sodium saccharine, sodium cyclamate, aspartame, acesulfame or a combination thereof.

20. The stable liquid pharmaceutical composition according to claim 1, wherein the bitter blocker is present, and wherein the bitter blocker comprises adenosine 5′-monophosphate, homoeriodictyol sodium, 30-hydroxydihydrocostunolide, gingerdione, or a combination thereof.

21. An stable oral suspension comprising:
(a) about 0.01% to about 5% w/v of lurasidone or a pharmaceutically acceptable salt thereof,
(b) at least one suspending agent;
(c) at least one buffering agent;
(d) at least one pharmaceutically acceptable vehicle; and
(e) optionally, at least one pharmaceutically acceptable excipient selected from preservatives, wetting agents, anti-foaming agents, anti-caking agents, bitter blockers, thickening agents, pH modifiers, stabilizers, anti-oxidants, chelating agents, flavoring agents, sweetening agents, coloring agents, or combination thereof, and
wherein, as measured using a USP Apparatus II at a paddle rotation speed of 50 RPM in 900 mL of a dissolution medium at 37±0.5° C., at least about 70 wt % of lurasidone dissolves within about 15 minutes in pH 3.8 McIlvaine buffer dissolution medium;
wherein the pH of the suspension ranges from about 2.0 to about 6.0; and
wherein the suspension is stable after being stored at about 45° C. and about 75% RH for at least 1 month.

22. The stable oral suspension according to claim 21, wherein the lurasidone or the pharmaceutically acceptable salt thereof has a D90 particle size of about 10 μm or less.

23. The stable oral suspension according to claim 21, wherein the suspension is a ready-to-use composition that does not need further reconstitution prior to administration.

24. The stable oral suspension according to claim 21, wherein the suspension comprises lurasidone or a pharmaceutical acceptable salt thereof at a concentration in a range of about 0.05 mg/mL to about 20 mg/mL.

25. The stable oral suspension according to claim 21, wherein the oral suspension comprises lurasidone or a pharmaceutical acceptable salt thereof at a concentration of about 10 mg/mL.
